SEQUENCE LISTING (30 sequences)

SEQ ID NO: 1 - DNA, 21 bp, synthetic construct
  source 1..21: mol_type = other DNA
gggcacatacgagtgtgttgt

SEQ ID NO: 2 - DNA, 23 bp, synthetic construct
  source 1..23: mol_type = other DNA
tcagctttgactgataacgtcac

SEQ ID NO: 3 - DNA, 22 bp, synthetic construct
  source 1..22: mol_type = other DNA
aaggggcaaaatggttctttcg

SEQ ID NO: 4 - DNA, 19 bp, synthetic construct
  source 1..19: mol_type = other DNA
gcacctgtatgtccccgag

SEQ ID NO: 5 - DNA, 23 bp, synthetic construct
  source 1..23: mol_type = other DNA
ctgctcatctatacacggttacc

SEQ ID NO: 6 - DNA, 22 bp, synthetic construct
  source 1..22: mol_type = other DNA
ggaaacgtcgtacagttctgtg

SEQ ID NO: 7 - DNA, 21 bp, synthetic construct
  source 1..21: mol_type = other DNA
ttggggtcaagcagattgcta

SEQ ID NO: 8 - DNA, 22 bp, synthetic construct
  source 1..22: mol_type = other DNA
gcagatgacacattggagaaga

SEQ ID NO: 9 - DNA, 20 bp, synthetic construct
  source 1..20: mol_type = other DNA
cagttcgtgaggttcgacag

SEQ ID NO: 10 - DNA, 19 bp, synthetic construct
  source 1..19: mol_type = other DNA
cagccgtacatgctctgga

SEQ ID NO: 11 - DNA, 23 bp, synthetic construct
  source 1..23: mol_type = other DNA
agtccctgtgctaggatttttca

SEQ ID NO: 12 - DNA, 22 bp, synthetic construct
  source 1..22: mol_type = other DNA
acataaactcgcctgattggtc

SEQ ID NO: 13 - DNA, 23 bp, synthetic construct
  source 1..23: mol_type = other DNA
tcaagcagtattggaacagagga

SEQ ID NO: 14 - DNA, 20 bp, synthetic construct
  source 1..20: mol_type = other DNA
caggaggctgcggacttttt

SEQ ID NO: 15 - DNA, 21 bp, synthetic construct
  source 1..21: mol_type = other DNA
tgaggtcacggacgattacat

SEQ ID NO: 16 - DNA, 22 bp, synthetic construct
  source 1..22: mol_type = other DNA
gtaggcccacgaaacaaatgat

SEQ ID NO: 17 - DNA, 23 bp, synthetic construct
  source 1..23: mol_type = other DNA
atgatggcttattacagtggcaa

SEQ ID NO: 18 - DNA, 20 bp, synthetic construct
  source 1..20: mol_type = other DNA
gtcggagattcgtagctgga

SEQ ID NO: 19 - DNA, 21 bp, synthetic construct
  source 1..21: mol_type = other DNA
gaccccaaggaaaactgggtg

SEQ ID NO: 20 - DNA, 22 bp, synthetic construct
  source 1..22: mol_type = other DNA
tgcttgaagtttcactggcatc

SEQ ID NO: 21 - DNA, 20 bp, synthetic construct
  source 1..20: mol_type = other DNA
tgcccattgaggtcatggtg

SEQ ID NO: 22 - DNA, 20 bp, synthetic construct
  source 1..20: mol_type = other DNA
cttgggtgggtcaggtttga

SEQ ID NO: 23 - DNA, 20 bp, synthetic construct
  source 1..20: mol_type = other DNA
tccccagggacctctctcta

SEQ ID NO: 24 - DNA, 22 bp, synthetic construct
  source 1..22: mol_type = other DNA
gggtttgctacaacatgggcta

SEQ ID NO: 25 - DNA, 23 bp, synthetic construct
  source 1..23: mol_type = other DNA
aggttcagtgttacccctcatca

SEQ ID NO: 26 - DNA, 22 bp, synthetic construct
  source 1..22: mol_type = other DNA
caccaccagggccatcattaaa

SEQ ID NO: 27 - DNA, 23 bp, synthetic construct
  source 1..23: mol_type = other DNA
tcggtaactgacttgaatgtcca

SEQ ID NO: 28 - DNA, 21 bp, synthetic construct
  source 1..21: mol_type = other DNA
tcgcttccctgttttagctgc

SEQ ID NO: 29 - DNA, 20 bp, synthetic construct
  source 1..20: mol_type = other DNA
agagctacgagctgcctgac

SEQ ID NO: 30 - DNA, 20 bp, synthetic construct
  source 1..20: mol_type = other DNA
agcactgtgttggcgtacag

(19) United States
(12) Patent Application Publication
(10) Pub. No.: US 2026/0109703 A1
(43) Pub. Date: Apr. 23, 2026

(54) OXYGEN-ECONOMIZED PHOTODYNAMIC THERAPY WITH NITRIC OXIDE-RELEASING PHOTOSENSITIZERS

(71) Applicant: City University of Hong Kong, Hong Kong (CN)

(72) Inventors: Pui Chi LO, Hong Kong (CN); Feijie XU, Shenzhen (CN); Kwan Ting CHOW, Hong Kong (CN); Meijun WANG, Hong Kong (CN); Eunice DOTSE, Hong Kong (CN)

(21) Appl. No.: 18/923,807

(22) Filed: Oct. 23, 2024

(51) Int. Cl. C07D 487/22; A61K 41/00; A61N 5/06

(52) U.S. Cl. CPC C07D 487/22; A61K 41/0071; A61N 5/062; A61N 2005/0663

(57) ABSTRACT

The subject invention relates to a novel compound and method for treating cancer in a subject using photodynamic therapy (PDT). The novel compound comprises zinc(II) phthalocyanine substituted with two or four nitric oxide (NO)-releasing moieties that release NO upon interaction with the intracellular glutathione, which reduces the cellular oxygen consumption rate, thus relieving the hypoxic status of cancer cells. Upon light irradiation, the novel compound generates reactive oxygen species (ROS), induces cytotoxicity under a hypoxic condition, overcoming the oxygen-dependent nature of PDT, and elicits the release of damage-associated molecular patterns, inducing the maturation of dendritic cells and triggering an antitumor immune response.

### SEQUENCE LISTING

The Sequence Listing for this application is labeled “CITY-101-SeqList.xml” which was created on Oct. 2, 2024 and is 27,149 bytes. The entire contents of the sequence listing is incorporated herein by reference in its entirety.

### BACKGROUND OF THE INVENTION

Photodynamic therapy (PDT) utilizes reactive oxygen species (ROS) for the elimination of malignant cells and tissues. These highly cytotoxic species are generated through the excitation of photosensitizers by light, followed by the interaction with the endogenous oxygen. This modality is a clinically approved procedure for the treatment of a variety of localized and superficial cancers.^{[1]} The treatment outcome depends largely on the tumor specificity of the photosensitizers, the oxygen concentration in the tumor microenvironment, and the efficiency of light delivery. Various approaches have been explored extensively with a view to confining the photodynamic action at the tumor site, thus reducing the unwanted photodamage.^{[2]} In particular, the active-targeting and activatable strategies have been integrated to enhance the specificity of the treatment.^{[3]} Recently, considerable efforts have been devoted to advancing the functions of photosensitizers and addressing the challenges of PDT, aiming to improve the tumor specificity, enhance the PDT efficacy against tumor hypoxia, and devise effective strategies to circumvent the limitation of light penetration.^{[4]}

It has been well documented that the efficacy of PDT is restricted by its dependence on oxygen, limiting its application in combating hypoxic tumors. Hypoxia is a common feature of advanced solid tumors arising from the rapid growth of cancer cells and the disrupted vasculatures, leading to inadequate oxygen delivery to the tissues.^{[5]} Moreover, oxygen is continuously depleted during the photodynamic process, which further exacerbates the tumor hypoxia and reduces the PDT efficacy. To address this challenge, various approaches have been investigated to enrich the oxygen level within the tumor microenvironment.^{[6]} For example, carriers such as hemoglobin and perfluorocarbons have been used to deliver oxygen to the tumor site to provide additional oxygen for PDT.^{[7]} However, these carriers suffer from poor oxygen loading, rapid oxygen leakage, difficulty in co-delivery of other therapeutics, and complex factors affecting the oxygen release. Another strategy involves the in-situ generation of oxygen, which can be achieved through catalytic transformation of endogenous H₂O₂ into oxygen by different means.^{[8]} However, these approaches are limited by the low endogenous H₂O₂ level, low catalytic efficiency, and the potential toxicity of the metal catalysts. Recently, an alternative strategy has been proposed that aims at inhibiting the cellular respiration in order to spare more endogenous oxygen for the photodynamic action. A wide range of agents, such as nitric oxide (NO),^{[9]} lonidamine,^{[10]} atovaquone (ATO),^{[11]} tamoxifen,^{[12]} metformin,^{[13]} and 7-amino carboxycoumarins-2^{[14]} have been used for this purpose. Cellular respiration, also known as mitochondria-associated oxidative phosphorylation (OXPHOS), consumes most of the cell's physiological oxygen.^{[15]} It has been reported that reducing the oxygen consumption is more effective than increasing the oxygen supply in alleviating tumor hypoxia.^{[16]}

Among the aforementioned agents, NO is of particular interest. It can inhibit the enzymatic function of cytochrome oxidase (complex IV) and suppress cellular respiration.^{[17]} Therefore, it is a promising agent for combating tumor hypoxia. To date, several classes of NO donors have been developed, such as organic nitrates, metal-NO complexes (e.g., sodium nitroprusside, SNP), N-nitrosamines, and nitrosothiols.^{[18]} NO donors are usually encapsulated in nanocarriers based on polymers or metal-organic frameworks.^{[9]} However, the fabrication of these nanomaterials usually involves a tedious protocol, and the nanomaterials may also pose potential hazard to healthy tissues. While most of the oxygen-economized photosensitizing systems, including those containing NO donors, are in a nanostructured form, the molecular counterparts have rarely been reported. Therefore, there is a need for effective and reliable agents that alleviate tumor hypoxia for enhanced PDT.

### BRIEF SUMMARY OF THE INVENTION

Photodynamic therapy (PDT) utilizes reactive oxygen species (ROS) for eradication of cancer cells. Its effectiveness is governed by the oxygen content, which is scarce in the hypoxic tumor microenvironment. In one aspect, the subject invention discloses a novel compound of formula I, obtained from a zinc(II) phthalocyanine substituted with two or four nitric oxide (NO)-releasing moieties, which suppresses the mitochondrial respiration, thereby sparing more intracellular oxygen for PDT.

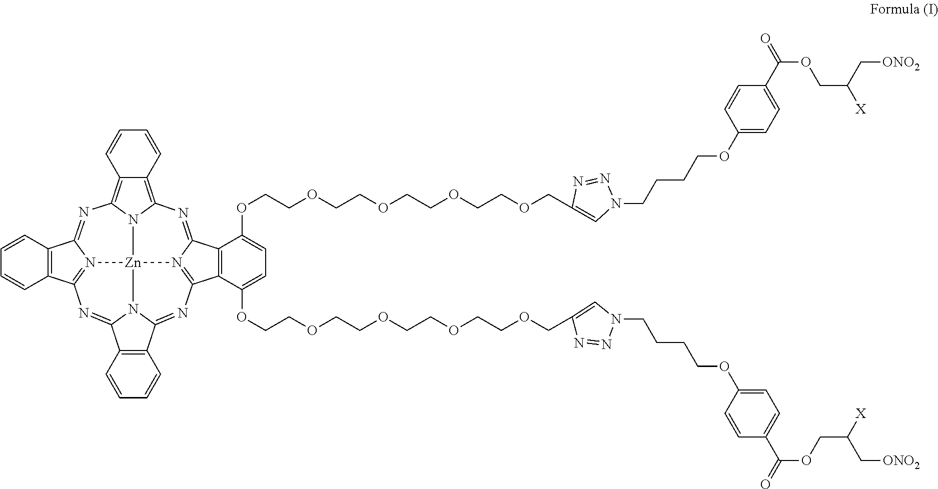

where x is H for compound ZnPc-2NO; and
where x is ONO₂ for compound ZnPc-4NO.

In another aspect, the subject invention relates to a novel pharmaceutical composition, comprising a compound having formula I, or pharmaceutically acceptable salt thereof, and one or more pharmaceutical carriers or excipients. In preferred embodiments, the composition comprises ZnPc-2NO and/or ZnPc-4NO, and any combination thereof.

In a further aspect, the subject invention relates to a method of treating a cancer with PDT in a subject in need thereof, comprising: (a) providing a composition comprising a compound of formula I, or pharmaceutically acceptable salt thereof, and one or more pharmaceutical carriers or excipients; (b) administering an effective amount of the composition to the cancer cells in the subject; and (c) activating the compound of formula I with light irradiation, where the activated compound induces cytotoxicity in cancer cells under normoxic and/or hypoxic condition.

In certain embodiments, the compound of the subject invention, releases NO upon interaction with the intracellular glutathione, which reduces the cellular oxygen consumption rate and adenosine triphosphate generation and alters the mitochondrial membrane potential. In certain embodiments, the compound relieves the hypoxic status of cancer cells and decreases the expression of hypoxia-inducible factor protein HIF-1α.

In certain embodiments, upon light irradiation, the compound generates ROS and induces cytotoxicity, under normoxic and hypoxic condition, overcoming the oxygen-dependent nature of PDT. In preferred embodiments, the photodynamic action of the compound elicits the release of damage-associated molecular patterns, inducing the maturation of dendritic cells and triggering an antitumor immune response.

### BRIEF DESCRIPTION OF THE DRAWINGS

FIG. 1 illustrates the mechanistic actions of the oxygen-economized PDT using ZnPc-2NO and the induced ICD. Upon internalization into cancer cells, ZnPc-2NO reacts with the enriched glutathione (GSH) to release NO, which interferes with the normal functions of mitochondria to retard the oxidative phosphorylation (OXPHOS), thereby reducing the oxygen consumption rate (OCR) and adenosine triphosphate (ATP) generation, eventually reversing the tumor hypoxic status and downregulating the hypoxia-inducible factor 1α (HIF-1 α) level for enhanced PDT. The photodynamic action can also induce the release of damage-associated molecular patterns (DAMPs), including calreticulin (CRT), ATP, and high-mobility group box-1 (HMGB1), triggering the maturation of dendritic cells (DCs), and elicitation of an antitumor immune response.

FIGS. 2A-2D—FIG. 2A illustrates electronic absorption and FIG. 2B fluorescence (λₑₓ=610 nm) spectra of ZnPc-2NO, ZnPc-4NO, and 11 (1 μM) in PBS with 10% (v/v) of DMF and 1% (v/v) of Tween 80. FIG. 2C illustrates comparison of the rates of decay of DPBF (initial concentration=30 μM) with or without the presence of ZnPc-2NO, ZnPc-4NO, or 11 (1 μM) and light irradiation (λ>610 nm) in DMF, as indicated by the decrease of absorbance at 416 nm. FIG. 2D illustrates cumulative release of NO from ZnPc-2NO and ZnPc-4NO (250 μM) with or without the presence of GSH (1 or 5 mM) in DMF over a period of time, as determined by a colorimetric Griess assay.

FIGS. 3A-3I—FIG. 3A illustrates fluorescence images of HT29 cells after being incubated in the culture medium with or without 11, ZnPc-2NO, or ZnPc-4NO (1 or 2 μM) for different periods of time, followed by incubation with DAF-FM-DA (5 μM) for 30 min (λₑₓ=488 nm, λₑₘ=495-600 nm). The NO generation efficiency is reflected by the intracellular fluorescence intensity of DAF-FM-T generated. FIG. 3B illustrates mean fluorescence intensities of DAF-FM-T in HT29 cells after the above treatments as determined by flow cytometry (λₑₓ=488 nm, λₑₘ=505-545 nm). FIG. 3C illustrates changes in dissolved oxygen content in the culture medium containing HT29 cells after being incubated in the culture medium with or without 11, ZnPc-2NO, or ZnPc-4NO (1 or 2 μM) for 8 h over a period of 30 min. FIG. 3D illustrates relative ATP content of HT29 cells after being incubated in the culture medium with or without 11, ZnPc-2NO, or ZnPc-4NO (1 or 2 μM) for 12 h. FIG. 3E illustrates analysis of ΔΨₘ on HT29 cells upon incubation in the culture medium with or without 11, ZnPc-2NO, or ZnPc-4NO (1 or 2 μM) for 12 h, followed by staining with JC-1 (10 μg mL⁻¹) for 30 min (red fluorescence: λₑₓ=561 nm, λₑₘ=570-680 nm; green fluorescence: λₑₓ=488 nm, λₑₘ=495-550 nm). FIG. 3F illustrates hypoxic status of HT29 cells as reflected by the intracellular fluorescence intensity of ROS-ID. The cells were incubated in the culture medium under a normoxic (21% O₂) or hypoxic (1% O₂) condition or in the presence of 11, ZnPc-2NO, or ZnPc-4NO (1 or 2 μM) under a hypoxic condition for 12 h, followed by incubation with ROS-ID (5 μM) for 1 h (λₑₓ=561 nm, λₑₘ=570-680 nm). FIG. 3G illustrates mean fluorescence intensities of ROS-ID in HT29 cells after the above treatments as determined by flow cytometry (λₑₓ=561 nm, λₑₘ=564-606 nm). FIG. 3H illustrates HIF-1α immunostaining for HT29 cells after incubation in the culture medium under a normoxic or hypoxic condition or in the presence of 11, ZnPc-2NO, or ZnPc-4NO (1 or 2 μM) under a hypoxic condition for 12 h (λₑₓ=488 nm, λₑₘ=493-565 nm). FIG. 3I illustrates mean fluorescence intensities of HIF-1α immunostaining in HT29 cells after the above treatments as determined by flow cytometry (λₑₓ=488 nm, λₑₘ=493-565 nm). Data are expressed as the mean±standard deviation (SD) of three independent experiments, and scale bar=20 nm in the relevant figures.

FIGS. 4A-4G—FIG. 4A illustrates bright field, fluorescence and the merged images of HT29 cells after incubation in the culture medium with or without 11, ZnPc-2NO, or ZnPc-4NO (1 or 2 μM) under a normoxic (FIG. 4A) or hypoxic (FIG. 4B) condition for 12 h, followed by incubation with H₂DCFDA (10 μM) for 30 min and light irradiation (λ>610 nm, fluence rate=18 mW cm⁻²) for 1 min (λₑₓ=488 nm, λₑₘ=505-545 nm). Scale bar=20 nm. FIG. 4C illustrates quantified fluorescence intensities of the cells being treated as described above determined by flow cytometry. Data are expressed as the mean±SD of three independent experiments. FIGS. 4D-4E illustrates dark and FIGS. 4F-4G photo-cytotoxicities of 11, ZnPc-2NO, and ZnPc-4NO against HT29 cells under normoxia (FIGS. 4D-4F) or hypoxia (FIGS. 4E-4G) (λ>610 nm, 18 mW cm⁻², 1.08 J cm⁻²). Data are expressed as the mean±standard error of the mean (SEM) of three independent experiments, each performed in quadruplicate. *P<0.05, **P<0.01, ***P<0.001, ****P<0.0001.

FIGS. 5A-5F—FIG. 5A illustrates CRT, HMGB1, and ATP tests. FIG. 5B shows confocal microscopic images of HT29 cells with CRT staining (λₑₓ=488 nm, λₑₘ=493-550 nm; for Hoechst: λₑₓ=405 nm, λₑₘ=460-490 nm) after the treatment with ZnPc-2NO (2 μM) for 12 h followed by light irradiation (λ>610 nm, 18 mW cm⁻²) for 1 min. The light-irradiated non-ZnPc-2NO-treated HT29 cells were used as control. Scale bar=100 nm. The extracellular HMGB1 (FIG. 5C) and ATP levels (FIG. 5D) of HT29 cells after the aforementioned treatments determined by a HMGB1 ELISA kit and an ATP bioluminescence kit, respectively. FIG. 5E illustrates relative maturation of DCs induced by the ZnPc-2NO-treated HT29 cells with or without light irradiation using CD80 and CD86 as markers. FIG. 5F illustrates relative mRNA levels of various markers of mature DCs after the aforementioned treatments. Data have been normalized to the ‘Blank’ values and analyzed by the 2-ΔΔCt method using β-actin as a reference gene. *P<0.05, **P<0.01, ***P<0.001, ****P<0.0001.

FIGS. 6A-6D—FIG. 6A is a schematic illustration of the experiment design to examine the antitumor immune response triggered by ZnPc-2NO-mediated PDT. FIG. 6B illustrates tumor growth curves for the ZnPc-2NO- and PBS-treated mice. FIG. 6C illustrates comparison of the tumor volumes of these two groups of mice on day 20. FIG. 6D illustrates change in the body weight of these two groups of mice over a period of 20 days. **P<0.01.

FIGS. 7A-7G illustrates population of innate immune cells in the lymph nodes on day 20, including leukocytes (FIG. 7A), neutrophils (FIG. 7B), macrophages (FIG. 7C), and NK cells (FIG. 7D), and the relative levels of mDCs (FIG. 7E), pDCs (FIG. 7F), and B cells (FIG. 7G) in the lymph nodes and tumor site on day 20. n.s. not significant, *P<0.05, **P<0.01, ****P<0.0001.

FIGS. 8A-8F illustrate populations of various T cells in the tumor site on day 20, including CD3⁺ T cells (FIG. 8A), CD4⁺ T cells (FIG. 8B), CD8⁺ T cells (FIG. 8C), and Tregs (FIG. 8D). FIG. 8E presents the ratio of CD8⁺ T cells to Tregs. FIG. 8F illustrates mean number of metastasized lung nodules on day 20. The statistical analysis of the p-value is also shown in each figure.

FIGS. 9A-9D illustrate electronic absorption (FIG. 9A-9C) and fluorescence emission (at 1 μM) (FIG. 9D) spectra of ZnPc-2NO, ZnPc-4NO, and 11 at different concentrations in DMF.

FIGS. 10A-10B—FIG. 10A illustrates fluorescence images and FIG. 10B the mean fluorescence intensities of HT29 cells after incubation with 11, ZnPc-2NO, or ZnPc-4NO (2 μM) for different periods of time as recorded using confocal microscopy (λₑₓ=633 nm, λₑₘ=650-900 nm) and flow cytometry (λₑₓ=638 nm, λₑₘ=700-725 nm), respectively. Scale bar=20 μm. Data are expressed as the mean±standard deviation (SD) of three independent experiments.

FIGS. 11A-11B—FIG. 11A illustrates fluorescence images and FIG. 11B the mean fluorescence intensities of A549 cells after incubation with 11, ZnPc-2NO, or ZnPc-4NO (2 μM) for different periods of time, as recorded using confocal microscopy (λₑₓ=633 nm, λₑₘ=650-900 nm) and flow cytometry (λₑₓ=638 nm, λₑₘ=700-725 nm), respectively. Scale bar=20 μm. Data are expressed as the mean±standard deviation (SD) of three independent experiments.

FIGS. 12A-12B—FIG. 12A illustrates fluorescence images of A549 cells after being incubated in the culture medium with or without 11, ZnPc-2NO, or ZnPc-4NO (1 or 2 μM) for different periods of time, followed by incubation with DAF-FM-DA (5 μM) for 30 min (λₑₓ=488 nm, λₑₘ=495-600 nm). Scale bar=10 μm. FIG. 12B illustrates mean fluorescence intensities of DAF-FM-T in A549 cells after the above treatments as determined by flow cytometry (λₑₓ=488 nm, λₑₘ=505-545 nm). Data are expressed as the mean±SD of three independent experiments.

FIG. 13 illustrates change in dissolved oxygen content in the culture medium containing A549 cells after being incubated in the culture medium with or without 11, ZnPc-2NO, or ZnPc-4NO (1 or 2 μM) for 8 h over a period of 30 min. Data are expressed as the mean±SD of three independent experiments.

FIG. 14 illustrates relative ATP content of A549 cells after being incubated in the culture medium with or without 11, ZnPc-2NO, or ZnPc-4NO (1 or 2 μM) for 12 h. Data are expressed as the mean±SD of three independent experiments.

FIG. 15 illustrates analysis of ΔΨₘ on A549 cells upon incubation in the culture medium with or without 11, ZnPc-2NO, or ZnPc-4NO (1 or 2 μM) for 12 h, followed by staining with JC-1 (10 μg mL⁻¹) for 30 min (red fluorescence: λₑₓ=561 nm, λₑₘ=570-680 nm; green fluorescence: λₑₓ=488 nm, λₑₘ=495-550 nm). Scale bar=10 nm.

FIGS. 16A-16B—FIG. 16A illustrates hypoxic status of A549 cells as reflected by the intracellular fluorescence intensity of ROS-ID. The cells were incubated in the culture medium under a normoxic (21% O₂) or hypoxic (1% O₂) condition or in the presence of 11, ZnPc-2NO, or ZnPc-4NO (1 or 2 μM) under a hypoxic condition for 12 h, followed by incubation with ROS-ID (5 μM) for 1 h (λₑₓ=561 nm, λₑₘ=570-680 nm). Scale bar=10 μm. FIG. 16B illustrates mean fluorescence intensities of ROS-ID in A549 cells after the above treatments as determined by flow cytometry (λₑₓ=561 nm, λₑₘ=564-606 nm). Data are expressed as the mean±SD of three independent experiments.

FIGS. 17A-17B—FIG. 17A illustrates HIF-1α immunostaining for A549 cells after incubation in the culture medium under a normoxic or hypoxic condition, or in the presence of 11, ZnPc-2NO, or ZnPc-4NO (1 or 2 μM) under a hypoxic condition for 12 h (λₑₓ=488 nm, λₑₘ=493-565 nm). Scale bar=10 μm. FIG. 17B illustrates mean fluorescence intensities of HIF-1α immunostaining in A549 cells after the above treatments as determined by flow cytometry (λₑₓ=488 nm, λₑₘ=493-565 nm). Data are expressed as the mean±SD of three independent experiments.

FIGS. 18A-18C illustrate bright field, fluorescence, and the merged images of A549 cells after incubation in the culture medium with or without 11, ZnPc-2NO, or ZnPc-4NO (1 or 2 μM) under a normoxic (FIG. 18A) or hypoxic (FIG. 18B) condition for 12 h, followed by incubation with H₂DCFDA (10 μM) for 30 min and light irradiation (λ>610 nm, fluence rate=18 mW cm⁻²) for 1 min (λₑₓ=488 nm, λₑₘ=505-545 nm). Scale bar=10 nm. FIG. 18C illustrates quantified fluorescence intensities of the cells being treated as described above determined by flow cytometry. Data are expressed as the mean±SD of three independent experiments.

FIGS. 19A-19D—(FIGS. 19A and 19B) Dark- and (FIGS. 19C and 19D) photo-cytotoxicities of 11, ZnPc-2NO, and ZnPc-4NO against A549 cells under normoxia (FIGS. 19A and 19C) or hypoxia (FIGS. 19B and 19D) (λ>610 nm, 18 mW cm⁻², 1.08 J cm⁻²). Data are expressed as the mean±SEM of three independent experiments, each performed in quadruplicate.

FIGS. 20A-20C illustrate confocal microscopic images of HT29 cells with CRT staining (λₑₓ=488 nm, λₑₘ=493-550 nm; for Hoechst: λₑₓ=405 nm, λₑₘ=460-490 nm) after the treatment with 11, ZnPc-2NO, or ZnPc-4NO (2 μM) for 12 h followed by light irradiation (λ>610 nm, 18 mW cm⁻²) for 1 min (FIG. 20A). Scale bar=100 μm. The extracellular HMGB1 (FIG. 20B) and ATP levels (FIG. 20C) of HT29 cells after the incubation with 11, ZnPc-2NO, or ZnPc-4NO (2 μM) for 12 h determined by a HMGB1 ELISA kit and an ATP bioluminescence kit, respectively. The data for the dark control are also included for comparison.

FIG. 21 illustrates ¹H (top) and ¹³C{¹H} (bottom) NMR spectra of 3 in DMSO-d₆.

FIG. 22 illustrates ¹H (top) and ¹³C{¹H} (bottom) NMR spectra of 6 in CDCl₃.

FIG. 23 illustrates ESI mass spectrum of 6 (top) and the enlarged region showing the molecular ion signal (bottom).

FIG. 24 illustrates ¹H (top) and ¹³C{¹H} (bottom) NMR spectra of 7 in CDCl₃.

FIG. 25 illustrates ESI mass spectrum of 7 (top) and the enlarged region showing the molecular ion signal (bottom).

FIG. 26 illustrates ¹H (top) and ¹³C{¹H} (bottom) NMR spectra of 10 in CDCl₃.

FIG. 27 illustrates ESI mass spectrum of 10.

FIG. 28 illustrates ¹H (top) and ¹³C{¹H} (bottom) NMR spectra of 11 in CDCl₃.

FIG. 29 illustrates ESI mass spectrum of 11 (top) and the enlarged region showing the molecular ion signal (bottom).

FIG. 30 illustrates ¹H (top) and ¹³C{¹H} (bottom) NMR spectra of ZnPc-2NO in CDCl₃.

FIG. 31 illustrates ESI mass spectrum of ZnPc-2NO (top) and the enlarged region showing the molecular ion signal (bottom).

FIG. 32 illustrates ¹H (top) and ¹³C{¹H}(bottom) NMR spectra of ZnPc-4NO in CDCl₃.

FIG. 33 illustrates ESI mass spectrum of ZnPc-4NO (top) and the enlarged region showing the molecular ion signal (bottom).

FIG. 34 illustrates the synthetic route for compounds ZnPc-2NO and ZnPc-4NO.

DESCRIPTION OF SEQUENCES
SEQ ID NO. 1:
5′-GGGCACATACGAGTGTGTTGT-3′
CD80 Forward
SEQ ID NO. 2:
3′-TCAGCTTTGACTGATAACGTCAC-5′
CD80 Reverse
SEQ ID NO. 3:
5′-AAGGGGCAAAATGGTTCTTTCG-3′
CD83 Forward
SEQ ID NO. 4:
3′-GCACCTGTATGTCCCCGAG-5′
CD83 Reverse
SEQ ID NO. 5:
5′-CTGCTCATCTATACACGGTTACC-3′
CD86 Forward
SEQ ID NO. 6:
3′-GGAAACGTCGTACAGTTCTGTG-5′
CD86 Reverse
SEQ ID NO. 7:
5′-TTGGGGTCAAGCAGATTGCTA-3′
CD40 Forward
SEQ ID NO. 8:
3′-GCAGATGACACATTGGAGAAGA-5′
CD40 Reverse
SEQ ID NO. 9:
5′-CAGTTCGTGAGGTTCGACAG-3′
MHC-I (HLA-B) Forward
SEQ ID NO. 10:
3′-CAGCCGTACATGCTCTGGA-5′
MHC-I (HLA-B) Reverse
SEQ ID NO. 11:
5′-AGTCCCTGTGCTAGGATTTTTCA-3′
MHC-II Forward
SEQ ID NO. 12:
3′-ACATAAACTCGCCTGATTGGTC-5′
MHC-II Reverse
SEQ ID NO. 13:
5′-TCAAGCAGTATTGGAACAGAGGA-3′
CD209 Forward
SEQ ID NO. 14:
3′-CAGGAGGCTGCGGACTTTTT-5′
CD209 Reverse
SEQ ID NO. 15:
5′-TGAGGTCACGGACGATTACAT-3′
CCR7 Forward
SEQ ID NO. 16:
3′-GTAGGCCCACGAAACAAATGAT-5′
CCR7 Reverse
SEQ ID NO. 17:
5′-ATGATGGCTTATTACAGTGGCAA-3′
IL-1β Forward
SEQ ID NO. 18:
3′-GTCGGAGATTCGTAGCTGGA-5′
IL-1β Reverse
SEQ ID NO. 19:
5′-GACCCCAAGGAAAACTGGGTG-3′
CXCL8 Forward
SEQ ID NO. 20:
3′-TGCTTGAAGTTTCACTGGCATC-5′
CXCL8 Reverse
SEQ ID NO. 21:
5′-TGCCCATTGAGGTCATGGTG-3′
IL-12p40 Forward
SEQ ID NO. 22:
3′-CTTGGGTGGGTCAGGTTTGA-5′
IL-12p40 Reverse
SEQ ID NO. 23:
5′-TCCCCAGGGACCTCTCTCTA-3′
TNFα Forward
SEQ ID NO. 24:
3′-GGGTTTGCTACAACATGGGCTA-5′
TNFα Reverse
SEQ ID NO. 25:
5′-AGGTTCAGTGTTACCCCTCATCA-3′
IFNA14 Forward
SEQ ID NO. 26:
3′-CACCACCAGGGCCATCATTAAA-5′
IFNA14 Reverse
SEQ ID NO. 27:
5′-TCGGTAACTGACTTGAATGTCCA-3′
IFNγ Forward
SEQ ID NO. 28:
3′-TCGCTTCCCTGTTTTAGCTGC-5′
IFNγ Reverse
SEQ ID NO. 29:
5′-AGAGCTACGAGCTGCCTGAC-3′
β-actin Forward
SEQ ID NO. 30:
3′-AGCACTGTGTTGGCGTACAG-5′
β-actin Reverse

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or λ±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. As used herein, the term “animal” may be, for example, humans, pigs, horses, goats, cats, dogs, apes, chimpanzees, orangutans, guinea pigs, hamsters, cows, or sheep. These benefits can include, but are not limited to, the treatment of a health condition, disease, or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the term “treatment” refers to eradicating, reducing, ameliorating, or reversing a sign or symptom of a health condition, disease, or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the terms “therapeutically-effective amount,” “therapeutically-effective dose,” “effective amount,” and “effective dose” are used to refer to an amount or dose of a compound or composition thereof that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the terms “arresting”, “reducing”, “inhibiting”, “blocking”, “preventing”, “alleviating”, or “relieving” when referring to a compound, mean that the compound brings down the occurrence, severity, size, volume or associated symptoms of cancer by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the cancer would normally progress without application of the compound or a composition comprising the compound.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

The recitation of a listing of chemical groups in any definition of a variable herein includes definitions of that variable as any single group or combination of listed groups. The recitation of an embodiment for a variable or aspect herein includes that embodiment as any single embodiment. Any compositions or methods provided herein can be combined with one or more of any of the other compositions and methods provided herein. or in combination with any other embodiments or portions thereof.

Other features and advantages of the invention will be apparent from the following description of the preferred embodiments thereof, and from the claims. All references cited herein are hereby incorporated by reference.

The subject invention pertains to a compound of formula I, or pharmaceutically acceptable salt thereof.

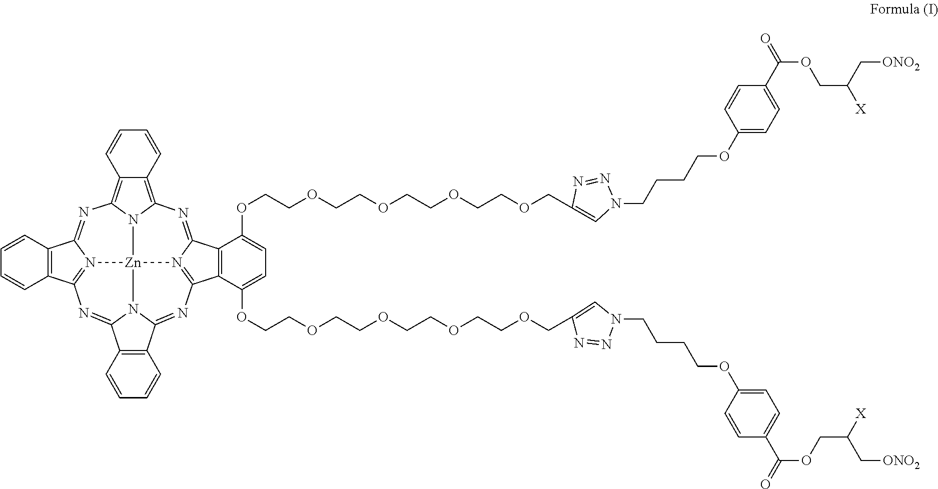

where x is H for compound ZnPc-2NO; and
where x is ONO₂ for compound ZnPc-4NO.

We designed a novel compound of formula I that comprises NO-releasing photosensitizers to alleviate cancer hypoxia for enhanced PDT. The compound comprises a zinc(II) phthalocyanine (ZnPc) core substituted with two or four glutathione (GSH)-responsive NO donors. Upon internalization into cancer cells, this compound reacts with the enriched intracellular GSH to release NO, which inhibits the cellular respiration, thereby reducing the oxygen consumption rate (OCR) and adenosine triphosphate (ATP) generation, eventually reversing the cancer hypoxic state.

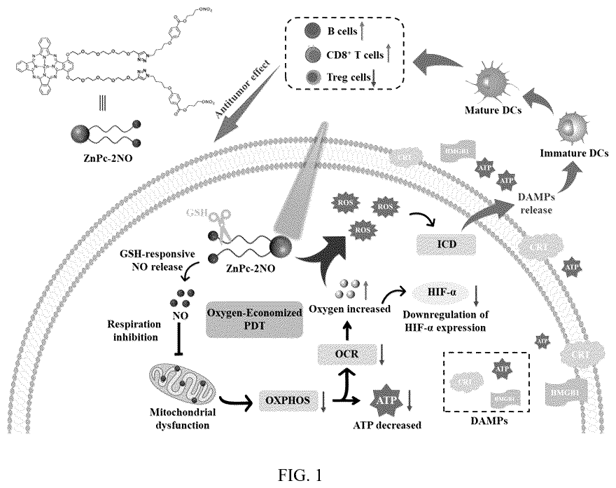

The mechanistic actions of the subject invention are outlined in FIG. 1, using ZnPc-2NO as an example. The photodynamic action of this photosensitizer induces immunogenic cell death (ICD) of cancer cells. It also induces the release of damage-associated molecular patterns (DAMPs), including calreticulin (CRT), ATP, and high-mobility group box-1 (HMGB1), and triggers the maturation of dendritic cells (DCs) and the production of pro-inflammatory cytokines.

In one aspect, the compound of formula I comprises two novel zinc(II) phthalocyanines, ZnPc-2NO and ZnPc-4NO, which have been designed for enhanced effectiveness in photodynamic therapy (PDT) for cancer treatment. ZnPc-2NO and ZnPc-4NO are substituted with two or four nitric oxide (NO)-releasing moieties, respectively, a feature that allows them to suppress mitochondrial respiration and conserve intracellular oxygen for PDT. Upon internalization into cancer cells, ZnPc-2NO and ZnPc-4NO react with intracellular glutathione and release NO, reducing the cellular oxygen consumption rate and adenosine triphosphate generation, and altering the mitochondrial membrane potential. This unique mechanism helps to relieve the hypoxic status of cancer cells and decrease the expression of hypoxia-inducible factor protein HIF-1α, for enhanced PDT.

In certain embodiments, upon light irradiation, the compound of the subject invention generates ROS and induces cytotoxicity under hypoxic conditions, thus overcoming a significant limitation of existing PDT technologies. The compound generates ROS and induces cytotoxicity also under normoxic conditions when irradiated. In certain embodiments, the compound causes ICD of the cancer cell. In preferred embodiments, the compound triggers an antitumor immune response by inducing the release of damage-associated molecular patterns and the maturation of dendritic cells.

In another aspect, disclosed herein is a pharmaceutical composition comprising a therapeutically effective amount of the compound of the subject invention, or pharmaceutically acceptable salt thereof, and one or more pharmaceutical carriers or excipients.

In a further aspect, the subject invention provides novel methods for treating a cancer with PDT in a subject afflicted by a malignant tumor, providing a composition comprising the compound of the subject invention, administering an effective amount of the composition to the tumor of the subject, and activating the compound via light irradiation, where the activated compound induces cytotoxicity in cancer cells under both normoxic and hypoxic condition. The activated compound releases NO in the cancer cells alleviating hypoxia by conserving intracellular oxygen for PDT, improving its effectiveness, especially in oxygen-scarce tumor environments. The compound further generates ROS and induces cytotoxicity under both normoxic and hypoxic condition.

In preferred embodiments, the compound is activated by light irradiation, preferably utilizing a laser, at an excitation frequency of about 610 to about 700 nm, and at a total fluence ranging from about 50 mW cm⁻² to about 300 mW cm⁻², for a time ranging from about 5 min to about 30 min for each treatment. In more preferred embodiments, the light irradiation is applied to cancer cells from about 3 to about 6 hours after administration of the composition. In certain embodiment, the composition is administered to the cancer cells as a series of treatments.

In certain embodiments, the compound triggers an antitumor immune response, causing ICD of cancer cells. In certain embodiments, the activated compound induces the release of DAMPs, including, but not limited to, CRT, ATP, and HMGB1. In preferred embodiments, the activated compound induces the maturation of dendritic cells and triggers an antitumor immune response. In certain embodiments, the antitumor immune response significantly increases the expression levels of cytokines, including, but not limited to, 1β (IL-1β), interleukin 10 (IL-10), interleukin 12 (IL-12p40), tumor necrosis factor α (TNF-α), IFN-α2, IFN-α14, and IFN-β.

In certain embodiments, the subject is a mammal. In preferred embodiments, the mammal is a human.

In certain embodiments, the malignant tumor or cancer is a superficial tumor or is a tumor accessible via a laser source coupled with an optical fiber.

In certain embodiments, the compositions of the subject invention can be formulated for parenteral administration e.g., by injection.

In one embodiment, the cell is a cancerous or tumorous cell, which is selected from, for example, prostate cancer cells, gallbladder cancer cells, intrahepatic biliary tract cancer cells, biliary tract cancer cells, oral cancer cells, pharyngeal cancer cells, laryngeal cancer cells, tongue cancer cells, duodenal cancer cells, eye tumor cells, mediastinal cancer cells, sinus cancer cells, renal pelvic cancer cells, heart cancer cells, glioblastoma cells, neuroblastoma cells, liver cancer cells, bone cancer cells, pancreatic cancer cells, skin cancer cells, head and neck cancer cells, breast cancer cells, lung cancer cells, skin or intraocular malignant melanoma cells, kidney cancer cells, uterine cancer cells, ovarian cancer cells, colon cancer cells, rectal cancer cells, anal region cancer cells, colorectal cancer cells, stomach cancer cells, testicular cancer cells, fallopian tube cancer endometrial carcinoma cells, cervical carcinoma cells, vaginal carcinoma cells, vulvar cancer cells, esophageal cancer cells, small intestinal cancer cells, endocrine system cancer cells, thyroid cancer cells, parathyroid cancer cells, adrenal gland cancer cells, soft tissue sarcoma cells, urethral cancer cells, penile cancer cells, childhood cancer cells, lymphocytic lymphoma cells, bladder cancer cells, ureter cancer cells, renal pelvic carcinoma cells, central nervous system (CNS) cancer cells, primary CNS lymphoma cells, spinal cancer cells, brainstem glioma cells, pituitary adenoma cells, Kaposi's sarcoma cells, epidermal cancer cells, squamous cell carcinoma cells, follicular lymphoma cells, immune large-cell lymphoma cells, mantle cell lymphoma cells, mycosis fungoides cells, hepatoblastoma cells, retinoblastoma cells, peritoneal cancer cells, brain tumor cells, thymic cancer cells, and any combination of the above cancer cells.

In some embodiments of the invention, the method comprises administration of multiple doses of the compositions of the subject invention. The method may comprise administration of therapeutically effective doses of a composition comprising the compound or composition thereof of the subject invention as described herein once a day, once a week, once a month, once a quarter, twice a year, once a year, or a lower frequency. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays to determine the presence of cancer cells, which are known in the art.

In some embodiments of the invention, the method comprises activating the compound of the subject invention with light irradiation. The method may comprise irradiating the cancer cells treated with the composition of the subject invention 1, 2, 3, 4, 5, 6, 7, 8, 9, 10, 15, 20, 25, 30, 35, 40, 50, 60, 70, 80, 90, 100 or more times. In some embodiments, the cancer is irradiated over the course of 1 day, 2 days, 3 days, 4 days, 5 days, 6 days, 7 days, 10 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks, about 1.5 years, about 2 years, about 2.5 years, about 5 years, or more than 10 years. Moreover, treatment of a subject with irradiation of the cancer can include a single treatment or can include a series of treatments. It will also be appreciated that the effective excitation frequency, intensity, and time used for treatment may increase or decrease over the course of a particular treatment. Changes in excitation frequency, intensity, and duration may result and become apparent from the results of testing for cancer remission well known in the art. In some embodiments of the invention, the method comprises irradiation of the compounds at several times per day, including but not limiting to 2 times per day, 3 times per day, and 4 times per day. In preferred embodiments, the irradiation of at least one dose of the composition is repeated every day for about 2 weeks to about 10 weeks.

In some embodiments, a cancer or tumor being targeted by the subject invention is in any of a variety of stages including newly diagnosed, relapsed, refractory, progressive disease, remission, and others. In some embodiments, the cancer or tumor being treated is a newly diagnosed cancer. In some embodiments, the cancer or tumor is a recurrent cancer (e.g., a recurrent gynecologic cancer such as recurrent epithelial ovarian cancer, recurrent fallopian tube cancer, recurrent primary peritoneal cancer, or recurrent endometrial cancer). In some embodiments, the present invention is applicable to treatment of metastatic cancers.

In certain embodiments, the therapeutically effective amount of the composition of the invention can be administered through intraperitoneal administration or by sustained release systems, such as semipermeable matrices of solid hydrophobic polymers containing the compounds of the invention. Administration may be also by way of other carriers or vehicles such as patches, micelles, liposomes, vesicles, implants (e.g. microimplants), synthetic polymers, microspheres, nanoparticles, and the like. In certain embodiments, the compositions may be administered using a nanoparticle to passage the composition through skin.

In certain embodiments, the compositions of the subject invention may be formulated for parenteral administration e.g., by injection, for example, bolus injection, intravenous administration, intraperitoneal administration, or continuous infusion. In addition, the compositions may be presented in unit dose form in ampoules, pre-filled syringes, and small volume infusion or in multi-dose containers with or without an added preservative. The compositions may be in forms of a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl phosphatidylcholine in USP WFI and 1 to 10% squalene or parenteral vegetable oil-in-water emulsions.

The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

Regardless of the route of administration selected, the composition may be formulated into pharmaceutically acceptable dosage form by conventional methods known to those of skill in the art. The composition may be formulated for administration in any convenient way for use in human or veterinary medicine, by analogy with other pharmaceuticals.

The compositions can further comprise one or more pharmaceutically acceptable carriers, and/or excipients, and can be formulated into preparations, for example, semi-solid or liquid forms, such as solutions or injections.

The formulations may conveniently be presented in unit dosage form and may be prepared any methods well known in the art of pharmacy. The amount of compound which can be combined with a carrier material to produce a single dosage form will vary depending upon the subject being treated, the particular mode of administration. The amount of an active ingredient which can be combined with carrier material to produce a single dosage form will usually be the amount of the compound which produces a therapeutic effect. Usually, out of one hundred percent, this amount will range from about 1 wt % to about 99 wt % of active ingredient, preferably from about 5 wt % to about 70 wt %, most preferably from about 10 wt % to about 30 wt %.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaternium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

### Materials and Methods

### General

All solvents and reagents were of HPLC or reagent grade and used as received. DMF and tetrahydrofuran (THF) were dried through routine protocols. All the reactions were monitored by thin layer chromatography (TLC) performed on pre-coated 0.20 mm silica gel 60 UV254 plates and visualized under UV light. Chromatographic purification was carried out on silica gel (Macherey-Nagel, 230-400 mesh) with the indicated eluent. ¹H and ¹³C{¹H} NMR spectra were recorded with a Bruker AVANCE III HD-300 or HD-400 spectrometer in deuterated solvents. Spectra were referenced internally using the residual solvent [¹H: δ=7.26 (for CDCl₃) or 2.49 (for DMSO-d₆)] or solvent [¹³C: δ=77.2 (for CDCl₃), 39.5 (for DMSO-d₆)] resonances relative to SiMe₄. The values of the chemical shifts are expressed in δ values (ppm) and the coupling constants (J) in Hz. ESI mass spectra were recorded on a Finnigan MAT 95 XL spectrometer. Electronic absorption and steady-state fluorescence spectra were taken on a Cary 5G UV-Vis-NIR spectrophotometer and a Hitachi F-7000 spectrofluorometer, respectively. The fluorescence quantum yields (Φ_{F}) of the samples (in DMF) were determined by the equation: Φ_{F}(sample)=(Fₛₐₘₚₗₑ/F_{ref})(A_{ref}/Aₛₐₘₚₗₑ)(n²ₛₐₘₚₗₑ/n²_{ref})Φ_{F(ref)}, where F, A, and n are the measured fluorescence (area under the emission peak), the absorbance at the excitation position (610 nm), and the refractive index of the solvent, respectively. The unsubstituted ZnPc in DMF was used as the reference [Φ_{F(ref)}=0.28].^{[19]} The singlet oxygen quantum yields (Φ_{Δ}) were measured in DMF by the method of chemical quenching of DPBF by using ZnPc as the reference (Φ_{Δ}=0.56).^{[20]}

Synthesis of Methyl 4-(4-azidobutoxy)benzoate (2)

To a solution of 1^{[21]} (1.50 g, 5.22 mmol) in DMSO (20 mL), sodium azide (1.70 g, 26.15 mmol) was added, and the mixture was stirred at room temperature for 5 h. It was then diluted with water and extracted with EtOAc. The organic solvent was washed with water for three times and then dried over anhydrous Na₂SO₄. The solvent was removed under reduced pressure to afford 2 (1.20 g), which was used for the next step without further purification.

Synthesis of 4-(4-Azidobutoxy)benzoic acid (3)

To a solution of crude 2 (1.10 g) in MeOH (10 mL), NaOH (0.40 g, 0.01 mol) in water (10 mL) was added slowly. The mixture was stirred under reflux for 2 h. MeOH was removed under reduced pressure, and the mixture was acidified with HCl (1 M). The precipitate formed was filtered off and purified by silica gel column chromatography using EtOAc/hexanes (1:1 v/v) as the eluent to afford 3 (0.95 g, 84% based on 1). ¹H NMR (300 MHz, DMSO-d₆): δ 12.64 (s, 1H, COOH), 7.88 (d, J=6.6 Hz, 2H, Ar—H), 7.02 (d, J=6.6 Hz, 2H, Ar—H), 4.07 (t, J=4.8 Hz, 2H, OCH₂), 3.42 (t, J=4.8 Hz, 2H, CH₂N₃), 1.76-1.81 (m, 2H, CH₂), 1.67-1.73 (m, 2H, CH₂). ¹³C{¹H} NMR (125.7 MHz, DMSO-d₆): δ 167.5, 162.6, 131.8, 123.4, 114.7, 67.7, 50.8, 26.3, 25.5. HRMS (ESI): m/z calcd for C₁₁H₁₃N₃NaO₃ [M+Na]⁺: 258.0849, found: 258.0850.

### Synthesis of Ester 6

EDCI (0.77 g, 4.96 mmol) and DMAP (61 mg, 0.50 mmol) were added to a mixture of 3 (0.95 g, 4.04 mmol) and 4^{[22]} (0.60 g, 5.0 mmol) in CH₂Cl₂. The mixture was stirred at room temperature overnight. It was then diluted with water and extracted with CH₂Cl₂. The organic layer was collected and dried under reduced pressure. The crude product was purified by silica gel column chromatography using EtOAc/hexanes (1:4 v/v) as the eluent to afford 6 (1.05 g, 77%). ¹H NMR (300 MHz, CDCl₃): δ 7.97 (d, J=8.7 Hz, 2H, Ar—H), 6.91 (d, J=8.7 Hz, 2H, Ar—H), 4.62 (t, J=6.3 Hz, 2H, OCH₂), 4.40 (t, J=6.3 Hz, 2H, OCH₂), 4.05 (t, J=6.0 Hz, 2H, OCH₂), 3.37 (t, J=6.6 Hz, 2H, CH₂N₃), 2.19 (quintet, J=6.3 Hz, 2H, CH₂), 1.65-1.95 (m, 4H, CH₂). ¹³C{¹H} NMR (100.6 MHz, CDCl₃): δ 166.1, 162.9, 131.7, 122.2, 114.1, 70.0, 67.4, 60.6, 51.1, 26.6, 26.4, 25.7. HRMS (ESI): m/z calcd for C₁₄H₁₈N₄NaO₆ [M+Na⁺]: 361.1119, found: 361.1117.

### Synthesis of Ester 7

EDCI (0.77 g, 4.96 mmol) and DMAP (61 mg, 0.50 mmol) were added into a mixture of 3 (0.95 g, 4.04 mmol) and 5^{[23]} (0.90 g, 4.97 mmol) in CH₂Cl₂. The mixture was stirred at room temperature overnight. It was then diluted with water and extracted with CH₂Cl₂. The organic layer was collected and dried under reduced pressure. The crude product was purified by silica gel column chromatography using EtOAc/hexanes (1:4 v/v) as the eluent to afford 7 (1.13 g, 70%). ¹H NMR (300 MHz, CDCl₃): δ 8.01 (d, J=8.7 Hz, 2H, Ar—H), 6.93 (d, J=8.7 Hz, 2H, Ar—H), 4.71 (t, J=5.1 Hz, 2H, OCH₂), 4.42-4.49 (m, 1H, OCH), 4.04 (t, J=5.1 Hz, 2H, OCH₂), 3.78-3.87 (m, 2H, OCH₂), 3.38 (t, J=6.3 Hz, 2H, CH₂N₃), 1.75-1.99 (m, 4H, CH₂). ¹³C{¹H} NMR (100.6 MHz, CDCl₃): δ 163.9, 162.3, 132.9, 121.2, 114.6, 76.0, 68.1, 67.6, 58.8, 51.1, 26.3, 25.7. HRMS (ESI): m/z calcd for C₁₄H₁₇N₅NaO₉ [M+Na]⁺: 422.0919, found: 422.0916.

### Synthesis of Phthalonitrile 10

To a solution of 2,3-dicyanohydroquinone (8) (0.50 g, 3.12 mmol) in DMF, alkynyl tosylate 9^{[24]} (2.41 g, 6.24 mmol) and K₂CO₃ (0.91 g, 6.58 mmol) were added. The mixture was stirred at 60° C. for 18 h. It was then diluted with water and extracted with EtOAc. The organic layer was collected and dried under reduced pressure. The crude product was purified by silica gel column chromatography using CH₂Cl₂/MeOH (60:1 v/v) as the eluent to afford 10 (1.30 g, 71%). ¹H NMR (400 MHz, CDCl₃): δ 7.27 (s, 2H, Ar—H), 4.22-4.25 (m, 4H, OCH₂), 4.18 (d, J=2.4 Hz, 4H, OCH₂), 3.87-3.89 (m, 4H, OCH₂), 3.72-3.75 (m, 4H, OCH₂), 3.63-3.70 (m, 20H, OCH₂), 2.44 (br s, 2H, C≡CH). ¹³C{¹H} NMR (100.6 MHz, CDCl₃): δ 155.4, 119.4, 113.1, 105.3, 79.6, 74.6, 71.9, 71.0, 70.6, 70.5, 70.4, 69.4, 69.1, 59.0, 58.4. HRMS (ESI): m/z calcd for C₃₀H₄₀N₂NaO₁₀ [M+Na]⁺: 611.2575, found: 611.2573.

### Synthesis of Phthalocyanine 11

A mixture of dialkynyl phthalonitrile 10 (0.20 g, 0.34 mmol), phthalonitrile (0.44 g, 3.43 mmol), and Zn(OAc)₂·2H₂O (0.75 g, 3.42 mmol) in n-pentanol (30 mL) was heated to 100° C., and then lithium (23 mg, 3.31 mmol) was added. The mixture was stirred at 110-120° C. for 4 h. After cooling, the volatiles were removed under reduced pressure. The residue was purified by flash silica gel column chromatography using CH₂Cl₂/MeOH (30:1 v/v) as the eluent, followed by size-exclusion chromatography using THF as the eluent. The crude product was further purified by silica gel column chromatography using CH₂Cl₂/MeOH (50:1 v/v) as the eluent to afford 11 (64 mg, 18%). ¹H NMR (300 MHz, CDCl₃ with a trace amount of pyridine-d₅): δ 9.30-9.41 (m, 6H, Pc-H_{α}), 8.06-8.11 (m, 6H, Pc-H_{β}), 7.46 (s, 2H, Pc-H_{β}), 4.90 (t, J=5.1 Hz, 4H, OCH₂), 4.49 (t, J=5.1 Hz, 4H, OCH₂), 4.09-4.12 (m, 8H, OCH₂), 3.82 (t, J=5.4 Hz, 4H, OCH₂), 3.66-3.69 (m, 4H, OCH₂), 3.58-3.63 (m, 12H, OCH₂), 2.35 (t, J=2.4 Hz, 2H, C≡CH). ¹³C{¹H} NMR (126 MHz, CDCl₃): δ 153.7, 153.5, 153.4, 153.0, 152.4, 150.1, 138.8, 138.4, 138.1, 128.9, 127.1, 122.6, 122.4, 114.7, 79.6, 74.6, 71.1, 70.8, 70.6, 70.5, 70.3, 69.0, 58.3 (some of the signals were overlapped). HRMS (ESI): m/z calcd for C₅₄H₅₂N₈NaO₁₀Zn [M+Na]⁺: 1059.2990, found: 1059.2989.

### Synthesis of ZnPc-2NO

To a solution of 11 (311 mg, 0.30 mmol) in THF (10 mL), compound 6 (100 mg, 0.30 mmol), CuI (76 mg, 0.40 mmol), and DIPEA (52 mg, 0.40 mmol) were added. The mixture was stirred at room temperature for 2 h, and then it was diluted with water and extracted with EtOAc. The organic layer was collected and dried under reduced pressure. The crude product was purified by silica gel column chromatography using CH₂Cl₂/MeOH (40:1 v/v) as the eluent to afford ZnPc-2NO (154 mg, 30%). ¹H NMR (400 MHz, CDCl₃): δ 9.29 (br s, 4H, Pc-H_{α}), 9.16 (br s, 2H, Pc-H_{α}), 8.03 (br s, 6H, Pc-H_{β}), 7.74 (d, J=8.4 Hz, 4H, Ar—H), 7.43 (s, 2H, triazole-H), 7.29 (br s, 2H, Pc-H_{β}), 6.61 (d, J=8.4 Hz, 4H, Ar—H), 4.81 (br s, 4H, CH₂), 4.48-4.52 (m, 8H, CH₂), 4.41 (br s, 4H, CH₂), 4.26 (t, J=6.0 Hz, 4H, CH₂), 4.19 (t, J=6.8 Hz, 4H, CH₂), 4.04-4.09 (m, 4H, CH₂), 3.78 (t, J=4.8 Hz, 4H, CH₂), 3.68 (t, J=6.0 Hz, 4H, CH₂), 3.62-3.65 (m, 4H, CH₂), 3.53-3.58 (m, 12H, CH₂), 2.06 (quintet, J=6.0 Hz, 4H, CH₂), 1.76-1.81 (m, 4H, CH₂), 1.49-1.53 (m, 4H, CH₂). ¹³C{¹H} NMR (126 MHz, CDCl₃): δ 166.0, 162.6, 154.3, 153.9, 149.6, 149.4, 149.1, 135.8, 135.6, 135.4, 131.6, 129.3, 123.5, 123.3, 123.1, 122.8, 122.6, 122.2, 114.0, 71.3, 70.9, 70.8, 70.78, 70.6, 70.1, 67.0, 60.7, 49.8, 27.0, 26.7, 26.0 (some of the signals were overlapped). HRMS (ESI): m/z calcd for C₈₂H₈₉N₁₆O₂₂Zn [M+H]⁺: 1715.5634, found: 1715.5654.

### Synthesis of ZnPc-4NO

To a solution of 11 (311 mg, 0.30 mmol) in THF (10 mL), compound 7 (134 mg, 0.34 mmol), CuI (76 mg, 0.40 mmol), and DIPEA (52 mg, 0.40 mmol) were added. The mixture was stirred at room temperature for 2 h, and then it was diluted with water and extracted with EtOAc. The organic layer was collected and dried under reduced pressure. The crude product was purified by silica gel column chromatography using CH₂Cl₂/MeOH (40:1 v/v) as the eluent to afford ZnPc-4NO (97 mg, 18%). ¹H NMR (400 MHz, CDCl₃): δ 9.45 (br s, 4H, Pc-H_{α}), 9.38 (br s, 2H, Pc-H_{α}), 8.14 (br s, 6H, Pc-H_{β}), 7.84 (d, J=8.4 Hz, 4H, Ar—H), 7.53 (br s, 2H, Pc-H_{β}), 7.43 (br s, 2H, triazole-H), 6.67 (d, J=8.4 Hz, 4H, Ar—H), 4.93 (br s, 4H, CH₂), 4.63-4.66 (m, 4H, CH₂), 4.52 (br s, 8H, CH₂), 4.40-4.44 (m, 2H, CH), 4.20 (br s, 4H, CH₂), 4.08-4.13 (m, 8H, CH₂), 3.78-3.86 (m, 8H, CH₂), 3.68-3.75 (m, 8H, CH₂), 3.59 (br s, 8H, CH₂), 1.85 (br s, 4H, CH₂), 1.56 (br s, 4H, CH₂). ¹³C{¹H} NMR (126 MHz, CDCl₃): δ 165.5, 162.8, 154.3, 149.5, 149.3, 149.1, 138.7, 135.8, 135.6, 135.4, 131.9, 129.4, 123.8, 122.8, 122.7, 121.8, 114.1, 71.3, 70.9, 70.8, 70.7, 70.6, 69.8, 69.5, 67.0, 65.4, 49.8, 47.4, 32.5, 29.9, 27.0, 26.0 (some of the signals were overlapped). HRMS (ESI): m/z calcd for C₈₂H₈₇N₁₈O₂₈Zn [M+H]⁺: 1837.5234, found 1837.5236.

### Singlet Oxygen Generation Measurements

The singlet oxygen generation efficiency was examined using DPBF as a scavenger. Briefly, a solution of ZnPc-2NO, ZnPc-4NO, or 11 in DMF (2 μM, 1.5 mL) was mixed with a solution of DPBF in DMF (60 μM, 1.5 mL). The resulting mixture ([ZnPc]=1 μM, [DPBF]=30 μM) was irradiated, and the absorbance at 416 nm was recorded after irradiation for every 5 s. The light source consisted of a 300 W halogen lamp, a water tank for cooling, and a color glass filter (Newport) cut-on at 610 nm. The fluence rate (>610 nm) was 18 mW cm⁻². The values of singlet oxygen quantum yield (Φ_{Δ}) were calculated as described previously.^{[20]}

### Indirect NO Measurements by Griess Assay

A solution of ZnPc-2NO or ZnPc-4NO in DMF (1 mM, 1 mL) was added to solution of GSH in DMF (1 or 5 mM, 3 mL). An aliquot of the resulting mixture (50 μL) was withdrawn and put into a well of a 96-well plate at different time points, and the released NO content was determined using a Griess reagent kit (Beyotime, no. S0021S). Briefly, solutions of Griess reagent I (containing sulphanilamide, 50 μL) and Griess reagent II (containing N-(1-naphthyl)ethylenediamine, 50 μL) were sequentially added to the mixture solution at room temperature. The absorption of the mixture at 540 nm was measured by a microplate reader. The NO concentration was calculated by measuring the absorbance at 540 nm and compared with the standard curve of NaNO₂ solution (1-100 μM). The release of NO from ZnPc-2NO and ZnPc-4NO (250 μM) was also examined in the absence of GSH for comparison.

### Cell Lines and Culture Conditions

HT29 human colorectal adenocarcinoma cells (ATCC, no. HTB-38) and A549 human lung adenocarcinoma cells (ATCC, no. CCL-185) were maintained in Roswell Park Memorial Institute (RPMI) 1640 medium (Thermo Fisher Scientific) supplemented with fetal bovine serum (FBS) (10% v/v) and penicillin-streptomycin (100 units mL⁻¹ and 100 μg mL⁻¹, respectively). 4T1 murine breast cancer cells (ATCC, no. CRL-2539) were maintained in Dulbecco's Modified Eagle Medium (DMEM) (Thermo Fisher Scientific) supplemented with FBS (10% v/v) and penicillin-streptomycin (100 units mL⁻¹ and 100 μg mL⁻¹, respectively). The cells were grown at 37° C. in a humidified 5% CO₂ atmosphere. To create a hypoxic condition, a chamber with 1% O₂, 5% CO₂, and 94% N₂ was used for cell incubation.

### Preparation of Stock Solutions

Appropriate amounts of 11, ZnPc-2NO, and ZnPc-4NO were dissolved in DMSO to form stock solutions with a concentration of 2 mM. These solutions were then diluted with PBS or the culture medium with 0.1% Tween 80 to the indicated concentrations for the in vitro studies. The final concentration of DMSO in these solutions was below the toxicity level (<0.1% v/v).

### Study of Cellular Uptake

Approximately 2×10⁵ HT29 and A549 cells in RPMI 1640 medium were seeded on a 35 mm confocal dish (MatTek Corporation, no. P35G-0-14-C) and incubated overnight at 37° C. in a humidified 5% CO₂ atmosphere. The medium was removed, and the cells were incubated in the medium with or without 11, ZnPc-2NO, or ZnPc-4NO (2 μM, 2 mL) for 2, 4, 6, or 12 h. After being washed with PBS for three times, the cells were examined with a Zeiss laser scanning microscope (Zeiss LSM880). The ZnPc was excited at 633 nm and its fluorescence was monitored at 650-900 nm. The images were digitized and analyzed using the Zen software. To further quantify the results, the cells were treated as described above, and then the cells were harvested and washed with PBS for three times. The fluorescence intensity of ZnPc was determined by flow cytometry (CytoFLEX S Beckman) with 10⁴ cells counted in each sample. The ZnPc was excited at 638 nm and its fluorescence was monitored at 700-725 nm, and the results were analyzed using the CytExpert software.

### Intracellular NO Release

The NO release inside the cells was detected using DAF-FM-DA (Beyotime, no. S0019) as the probe. Approximately 2×10⁵ HT29 and A549 cells in RPMI 1640 medium were seeded on a 35 mm confocal dish and incubated overnight at 37° C. in a humidified 5% CO₂ atmosphere. The medium was removed, and the cells were incubated in the medium with or without 11 (2 μM), ZnPc-2NO (2 μM), or ZnPc-4NO (1 or 2 μM) for 2, 6, or 12 h. After being washed with PBS for three times, the cells were treated with DAF-FM-DA (5 μM) in PBS at 37° C. for 30 min. The cells were then rinsed with PBS for three times and examined with a Zeiss laser scanning microscope (Zeiss LSM880). The benzotriazole fluorescein derivative DAF-FM-T was excited at 488 nm and its fluorescence was monitored at 495-600 nm. The images were digitized and analyzed using the Zen software. To further quantify the results, the cells were treated as described above using a 6-well plate. The cells were then harvested, and the fluorescence intensity of DAF-FM-T was determined by a flow cytometer (CytoFLEX S Beckman) with 10⁴ cells counted in each sample. The DAF-AM-T was excited at 488 nm and its fluorescence was monitored at 505-545 nm. The results were analyzed using the CytExpert software.

### Dissolved Oxygen Measurements

Approximately 1×10⁵ HT29 and A549 cells were seeded on a 6-well plate and cultured for 24 h. The medium was removed, and the cells were treated with 11 (2 μM), ZnPc-2NO (2 μM), or ZnPc-4NO (1 or 2 μM) in the medium for 8 h. The cells were washed with PBS for three times and then refed with the culture medium. The medium without the cells was used as the blank control, and the cells without the drug treatment were also used for comparison. The oxygen concentration was monitored by a dissolved oxygen meter (JPSJ-695F, INESA instruments, China) through immersing the electrode into the medium. During the measurements, the cell medium was sealed with liquid paraffin (2 mL) to avoid oxygen exchange. The dissolved oxygen value was recorded every 5 min over a period of 30 min. The relative dissolved oxygen content was calculated by the following equation and the experiment was repeated for 3 times. Dissolved oxygen (DO) (%)=(DO value at each time point/Basic DO value)×100%.

### Intracellular ATP Measurements

Approximately 1×10⁴ HT29 and A549 cells per well were seeded in a 96-well plate and incubated for 24 h. The medium was removed, and the cells were treated with 11 (2 μM), ZnPc-2NO (2 μM), or ZnPc-4NO (1 or 2 μM) for 12 h. The cells were washed with PBS for three times and then treated with a luminescent ATP detection kit (Abcam, no. ab113849) according to the protocol. Briefly, the cells were treated with the detergent solution (50 μL) at 4° C. for 5 min to lyse the cells and inactivate the ATP degrading enzymes. The substrate solution (50 μL) containing luciferase and luciferin was then added into the cells. After incubation at 4° C. in dark for 15 min, the luminescence was detected using a BioTek Synergy™ H1 microplate reader. The ATP content was calculated by comparing with the standard curve of ATP.

### Investigation of the Mitochondrial Membrane Potential (ΔΨm)

Approximately 2×10⁵ HT29 and A549 cells in RPMI 1640 medium were seeded on a 35 mm confocal dish and incubated overnight at 37° C. in a humidified 5% CO₂ atmosphere. The medium was removed, and the cells were incubated in the medium with or without 11 (2 μM), ZnPc-2NO (2 μM), or ZnPc-4NO (1 or 2 μM) for 12 h. After being washed with PBS for three times, the cells were treated with JC-1 (10 μg mL⁻¹, Thermo Fisher Scientific, no. T3168) in a serum-free medium and then incubated for a further 30 min in the dark. The medium was removed, and the cells were washed with PBS for three times. The fluorescence of the monomeric form (green: λₑₓ=488 nm, λₑₘ=495-550 nm) and the J-aggregate form (red: λₑₓ=561 nm, λₑₘ=570-680 nm) of JC-1 was examined by a Zeiss laser scanning microscope (Zeiss LSM880). The images were digitized and analyzed using the Zen software.

### Intracellular Hypoxic Levels

Approximately 2×10⁵ HT29 and A549 cells in RPMI 1640 medium were seeded on a 35 mm confocal dish and incubated overnight at 37° C. in a humidified 5% CO₂ atmosphere. The medium was removed, and the cells were incubated in the medium with or without 11 (2 μM), ZnPc-2NO (2 μM), or ZnPc-4NO (1 or 2 μM) for 12 h under hypoxia (1% O₂). The cells were rinsed with PBS for three times and then treated with the fluorogenic hypoxia probe ROS-ID (5 μM) in the medium (2 mL). The cells were then incubated for a further 1 h. Afterward, the medium was removed, and the cells were washed with PBS for three times and then examined using a Zeiss laser scanning microscope. The ROS-ID probe was excited at 561 nm and its fluorescence was monitored at 570-680 nm. The cells incubated under normoxia (21% O₂) without the drug treatment were used as control. The images were digitized and analyzed using the Zen software. To further quantify the results, the cells were treated as described above, and then the cells were harvested, and the fluorescence intensity of the hypoxia detection probe was determined by flow cytometry. The ROS-ID was excited at 561 nm and its fluorescence was collected at 564-606 nm. The results were digitized and analyzed using the CytExpert software.

### HIF-1α Immunostaining

Approximately 2×10⁵ HT29 and A549 cells in RPMI 1640 medium were seeded on a 35 mm confocal dish and incubated overnight at 37° C. in a humidified 5% CO₂ atmosphere. The medium was removed, and the cells were incubated in the medium with or without 11 (2 μM), ZnPc-2NO (2 μM), or ZnPc-4NO (1 or 2 μM) for 12 h under hypoxia. The cells incubated under normoxia without the drug treatment were used for comparison. The medium was removed, and the cells were rinsed with PBS twice. The cells were then fixed with 4% formaldehyde for 10 min, permeabilized in 0.1% Triton X-100 for 5 min, and then blocked in a solution containing 1% bovine serum albumin (BSA) in PBS for 1 h. Finally, the fixed cells were stained with the antibody against HIF-1α (Abcam, no. ab190197) for 1 h at room temperature. After being washed with PBS, the cells were examined with a Zeiss laser scanning microscope. The HIF-1α antibody was excited at 488 nm and its fluorescence was monitored at 493-565 nm. The images were digitized and analyzed using the Zen software. To further quantify the results, the cells were treated as described above. After fixing the cells with 4% formaldehyde for 15 min, the cells were resuspended in 90% of cold CH₃OH for 30 min at −20° C. After removing the CH₃OH, the cells were washed with 1% BSA in PBS and then incubated in this buffer for 30 min. Subsequently, the cells were stained with the antibody against HIF-1α for 1 h at room temperature. The cells were harvested and the fluorescence intensity of the antibody against HIF-1α was determined by flow cytometry. The antibody was excited at 488 nm and its fluorescence was monitored at 505-545 nm. The results were analyzed using the CytExpert software.

### Intracellular ROS Generation

Approximately 2×10⁵ HT29 and A549 cells in RPMI 1640 medium were seeded on a 35 mm confocal dish and incubated overnight at 37° C. in a humidified 5% CO₂ atmosphere. The medium was removed and the cells were incubated with 11 (2 μm), ZnPc-2NO (2 μM), or ZnPc-4NO (1 or 2 μM) for 12 h under a normoxic or hypoxic condition. The medium was removed and the cells were further incubated with H₂DCFDA (Sigma-Aldrich, 10 μM) for 30 min. The cells were irradiated with a 300 W halogen lamp after passing through a water tank for cooling and a color glass filter (Newport) cut-on at 610 nm. The fluence rate (λ>610 nm) was 18 mW cm⁻². Illumination of 1 min led to a total fluence of 1.08 J cm⁻². Finally, the cells were examined with a Zeiss laser scanning microscope. The oxidized product DCF was excited at 488 nm and its fluorescence was monitored at 493-600 nm. The images were digitized and analyzed using the Zen software. To quantify the intracellular fluorescence intensities of DCF, flow cytometry was performed after the above treatments and harvesting the cells. DCF was excited at 488 nm and its fluorescence was monitored at 505-545 nm. The results were analyzed using the CytExpert software.

### Study of Cytotoxicity

Approximately 1×10⁴ HT29 and A549 cells per well in RPMI 1640 medium were inoculated in 96-well plates and incubated for 24 h. The cells were treated with different concentrations of 11, ZnPc-2NO, or ZnPc-4NO ([ZnPc]=0.25, 0.5, 1, 2, and 4 μM) under a normoxic or hypoxic condition for 12 h. After being washed with PBS for three times, the cells were irradiated with the aforementioned light source for 1 min. The cells were then incubated overnight under normoxia or hypoxia. MTT assay was used to evaluate the cell viability. An MTT solution in PBS (3 mg mL⁻¹, 50 μL) was added to each well, followed by incubation for 4 h at 37° C. in a humidified 5% CO₂ atmosphere. After removing the MTT solution, DMSO (150 μL) was added to each well. The plate was agitated on a BioTek microplate reader at ambient temperature for 10 s before measuring the absorbance at 490 nm for each well. The average absorbance of the blank wells, which did not contain the cells, was subtracted from the readings of the other wells. The cell viability was determined by the following equation: % viability=[Σ(Aᵢ/A_{control})×100]/n, where Aᵢ is the absorbance of the i^{th} datum (i=1, 2, . . . , n), A_{control} is the average absorbance of the control wells in which no sample was treated, and n (=4) is the number of data points.

### Immunofluorescence Imaging of CRT

Approximately 2×10⁵ HT29 cells in RPMI 1640 medium were seeded on a 35 mm glass bottom dish and incubated overnight at 37° C. in a humidified 5% CO₂ atmosphere. The medium was removed and the cells were incubated in the neat medium or with 11, ZnPc-2NO, or ZnPc-4NO (2 μM) for 12 h. After being washed with PBS for three times, the cells were irradiated with the aforementioned light source for 1 min, followed by incubation for a further 6 h. The cells were then fixed with 4% formaldehyde for 10 min, permeabilized in 0.1% Triton X-100 for 5 min, and then blocked in a solution containing 1% BSA in PBS for 1 h. Finally, the fixed cells were stained with a primary antibody against CRT (Beyotime, No. AF2425) for 1 h at room temperature. After being washed with PBS, the cells were treated with the secondary antibody Alexa Fluor 488-labeled goat anti-Rabbit IgG (Beyotime, No. A0423) for a further 1 h. The cells were washed with PBS for three times and then incubated with Hoechst (Thermo Fisher Scientific, No. 62249, 20 μM) for 10 min. After being washed with PBS, the cells were examined with a Zeiss laser scanning microscope. The Alexa Fluor 488 was excited at 488 nm and its fluorescence was monitored at 493-550 nm. The Hoechst was excited at 405 nm and its fluorescence was monitored at 460-490 nm. The images were digitized and analyzed using the Zen software.

### Extracellular HMGB1 Measurements

Approximately 2×10⁵ HT29 cells in RPMI 1640 medium were seeded in a 6-well plate and incubated overnight at 37° C. in a humidified 5% CO₂ atmosphere. The medium was removed and the cells were incubated in the neat medium or with 11, ZnPc-2NO, or ZnPc-4NO (2 μM) for 12 h. After being washed with PBS for three times, the cells were irradiated with the aforementioned light source for 1 min. The cells without being irradiated were used as the dark control. After incubation for 12 h, the supernatants were collected for the measurement of extracellular HMGB1 content using a colorimetric ELISA kit (Fankew, No. F1616-B) according to the company's protocol. Briefly, 50 μL of the standard solutions, samples [a mixture of supernatant (10 μL) and diluent (40 μL)], and control (diluent only) were added into different wells of the plate. After incubation at 37° C. for 30 min, the liquids were removed, and the wells were washed with a washing buffer for 5 times. HRP-antibody substrate (50 μL) was then added into each well (except the control well) and the plate was further incubated at 37° C. for 30 min. After washing with a washing buffer for 5 times, reagent A (50 μL) and reagent B (50 μL) were added into each well (including the control). After incubation at 37° C. for 10 min, a stop solution (50 μL) was added into each well and the optical density of each well was measured at 450 nm using a microplate reader. The control was set as zero. The HMGB1 concentration of the supernatants was calculated using the standard curve of the absorbance at 450 nm against the concentration of HMGB1 standard solution.

### Extracellular ATP Measurements

Approximately 1×10⁵ HT29 cells per well were seeded in a 6-well plate and cultured for 24 h. After removal of the medium, the cells were incubated in the neat medium or with 11, ZnPc-2NO, or ZnPc-4NO (2 μM) for 12 h. After being washed with PBS for three times, the cells were irradiated with the aforementioned light source for 1 min. The cells without being irradiated were used as the dark control. After incubation for 12 h, the supernatants were collected for the measurement of extracellular ATP content using a luminescent ATP detection kit. Briefly, the supernatants were treated with the detergent solution (50 μL) at 4° C. for 5 min to deactivate the ATP degrading enzymes. The substrate solution (50 μL) containing luciferase and luciferin was then added into the supernatants, which was then incubated at 4° C. in dark for 15 min. The luminescence was detected using a BioTek Synergy™ H1 microplate reader. The ATP content was calculated using the standard curve of ATP.

Generation of Immature DCs (imoDCs) from THP-1 Cells

THP-1 cells at a concentration of 2×10⁵ cells mL⁻¹ were cultured and maintained in RPMI 1640 medium supplemented with penicillin-streptomycin, FBS (10%), recombinant human interleukin-4 (IL-4, PeproTech, no. 200-04, 100 ng mL⁻¹) and recombinant human granulocyte macrophage colony-stimulating factor (GM-CSF, PeproTech, no. 300-03, 100 ng mL⁻¹). The cells were cultured at 37° C. under 5% CO₂ for 6 days, and the culture medium was replaced with a fresh cytokine-supplemented medium every two days. The cells were harvested at the end of differentiation period by using StemPro™ Accutase Cell Dissociation Reagent (no. A1110501) for further co-culturing with HT29 cells.

### Study of DCs' Maturation Induced by PDT-Treated Cells

Approximately 1×10⁵ HT29 cells per well in RPMI 1640 medium were inoculated in a 6-well plate and incubated for 24 h. The cells were treated with ZnPc-2NO in the medium (2 μM) under a normoxic condition for 12 h. After being washed with PBS for three times, the cells were irradiated with the aforementioned light source. Illumination of 1 min led to a total fluence of 1.08 J cm⁻². The cells without being irradiated were treated as the dark control. After incubation for a further 12 h, HT29 cells were washed with PBS for three times and co-cultured with imoDCs at a ratio of 1:20 (imoDCs:HT29) for 24 h. The maturation of dendritic cells was determined by flow cytometry using PE anti-human CD80 antibody (BioLegend, No. 305208) and Brilliant Violet 510 anti-human CD86 antibody (BioLegend, No. 305432).

### Reverse Transcription Quantitative PCR (RT-qPCR) (a Cytokine Test)

Immune response-associated cytokines were determined by RT-qPCR. Briefly, about 1×10⁵ HT29 cells were incubated in RPMI 1640 medium with or without ZnPc-2NO (2 μM) for 12 h under a normoxic condition, followed by the aforementioned light treatment. The cells were then co-cultured with imoDCs at a ratio of 1:20 (imoDCs:HT29) for 24 h. Unstimulated imoDCs were used as a negative control. After detachment and removal of the imoDCs, the cells were lysed with TRIzol Reagent (Thermo Fisher, No. 15596018) by repeated pipetting. Total RNA was isolated according to the manufacturer's instruction and the cDNA was prepared using the PrimeScript™ RT Reagent Kit (Takara, No. RR036A) according to the manufacturer's instructions. The qPCR was performed using PowerUp™ SYBR™ Green Master Mix (Thermo Fisher, No. A25776) on a QuantStudio™ 5 real-time PCR system (Applied Biosystems). Gene-specific primers used are listed in Table S1. The data were analyzed by the ΔΔCT method and normalized to the β-actin expression level.

TABLE S1
Sequence of primers for qRT-PCR analysis

| Gene | Forward (5′ to 3′) | Reverse (5′ to 3′) |
|---|---|---|
| CD80 | GGGCACATACGAGTGTGTTGT | TCAGCTTTGACTGATAACGTCAC |
| | (SEQ ID NO. 1) | (SEQ ID NO. 2) |
| CD83 | AAGGGGCAAAATGGTTCTTTCG | GCACCTGTATGTCCCCGAG |
| | (SEQ ID NO. 3) | (SEQ ID NO. 4) |
| CD86 | CTGCTCATCTATACACGGTTACC | GGAAACGTCGTACAGTTCTGTG |
| | (SEQ ID NO. 5) | (SEQ ID NO. 6) |
| CD40 | TTGGGGTCAAGCAGATTGCTA | GCAGATGACACATTGGAGAAGA |
| | (SEQ ID NO. 7) | (SEQ ID NO. 8) |
| MHC-I | CAGTTCGTGAGGTTCGACAG | CAGCCGTACATGCTCTGGA |
| (HLA-B) | (SEQ ID NO. 9) | (SEQ ID NO. 10) |
| MHC-II | AGTCCCTGTGCTAGGATTTTTCA | ACATAAACTCGCCTGATTGGTC |
| | (SEQ ID NO. 11) | (SEQ ID NO. 12) |
| CD209 | TCAAGCAGTATTGGAACAGAGGA | CAGGAGGCTGCGGACTTTTT |
| | (SEQ ID NO. 13) | (SEQ ID NO. 14) |
| CCR7 | TGAGGTCACGGACGATTACAT | GTAGGCCCACGAAACAAATGAT |
| | (SEQ ID NO. 15) | (SEQ ID NO. 16) |
| IL-1β | ATGATGGCTTATTACAGTGGCAA | GTCGGAGATTCGTAGCTGGA |
| | (SEQ ID NO. 17) | (SEQ ID NO. 18) |
| CXCL8 | GACCCCAAGGAAAACTGGGTG | TGCTTGAAGTTTCACTGGCATC |
| | (SEQ ID NO. 19) | (SEQ ID NO. 20) |
| IL-12p40 | TGCCCATTGAGGTCATGGTG | CTTGGGTGGGTCAGGTTTGA |
| | (SEQ ID NO. 21) | (SEQ ID NO. 22) |
| TNFα | TCCCCAGGGACCTCTCTCTA | GGGTTTGCTACAACATGGGCTA |
| | (SEQ ID NO. 23) | (SEQ ID NO. 24) |
| IFNA14 | AGGTTCAGTGTTACCCCTCATCA | CACCACCAGGGCCATCATTAAA |
| | (SEQ ID NO. 25) | (SEQ ID NO. 26) |
| IFNγ | TCGGTAACTGACTTGAATGTCCA | TCGCTTCCCTGTTTTAGCTGC |
| | (SEQ ID NO. 27) | (SEQ ID NO. 28) |
| β-actin | AGAGCTACGAGCTGCCTGAC | AGCACTGTGTTGGCGTACAG |
| | (SEQ ID NO. 29) | (SEQ ID NO. 30) |

### In Vivo Antitumor Efficacy and Immune Response

Fourteen adult female BALB/c mice aged 6-8 weeks were purchased from Laboratory Animal Research Unit (LARU) of the City University of Hong Kong. The mice were housed in a laboratory environment at 24±1° C. under a 12 h dark-light cycle and allowed to acclimatize for 7 days before commencement of experiments. The protocols of animal experiments were approved by the Animal Experimentation Ethics Committee of the City University of Hong Kong. At about 8 weeks of age, 4T1 tumor cells (5×10⁵ cells in 100 μL) were injected into the mammary fat pad of female Balb/c mice. When the tumor reached a volume of about 50 mm³, the mice were treated with PBS or ZnPc-2NO in PBS with 1% (v/v) DMSO and 0.1% (v/v) Tween 80 (100 μM, 250 μL, 25 nmol) (1 nmol per gram body weight of mouse) intratumorally (n=7). After 3 h, the tumors were irradiated with a 675 nm laser at 0.1 W for 5 min. Illumination on a spot size of 1.0 cm² for 5 min led to a total fluence of 30 J cm⁻². Tumor growth was monitored over 20 days and the tumor size was measured using a digital caliper every 2 days. Tumor volume was calculated based on the following equation: tumor volume (mm³)=(length×width×width)/2. Body weight of the mice was recorded every 2 days. On the last day of the study, the mice were euthanized, and tumors were collected and weighed. Cells from the digested tumor tissues and lymph nodes were prepared for flow cytometric analysis.

Cells isolated from tumor and lymph nodes were blocked with TruStain FcX™ (anti-mouse CD16/32) antibody (BioLegend, No. 101319) and stained with the following antibodies, including FITC anti-mouse F4/80 (BioLegend, No. 123108), APC anti-mouse/human CD45R/B220 (BioLegend, No. 103212), APC/Cyanine 7 anti-mouse/human CD11b (BioLegend, No. 101226), Pacific Blue anti-mouse CD45 (BioLegend, No. 103126), PE anti-mouse CD314/NKG2D (BioLegend, No. 130207), Alexa Fluor 594 anti-mouse Ly-6G/Ly-6C/Gr-1 (BioLegend, No. 108448), PerCP/Cyanine 5.5 anti-mouse CD11c (BioLegend, No. 117328), PE/Cyanine anti-mouse CD3 (BioLegend, No. 100220), APC/Cyanine 7 anti-mouse CD25 (BioLegend, No. 102026), and Alexa Fluor 594 anti-mouse CD4 (BioLegend, No. 100446) according to manufacturer's instructions. After staining, about 2×10⁶ cells were analyzed using a flow cytometer (Beckman Coulter) with the CytExpert Software. Commercial beads (BioLegend, No. 424602) were used as compensation controls.

### Measurements of Metastasized Nodules

On day 20 after PDT, the mice were euthanized and the lungs were injected with India Ink via the trachea using a 1 mL syringe. After complete inflation of the lungs with ink, the lungs were lifted out of the mice and rinsed briefly with cold PBS. The lungs were then fixed in a Fekete's solution (10 mL) overnight. The number of metastasized tumor nodules on the lungs was counted as white nodules against a dark background by naked eye.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Molecular Design, Synthesis, and Characterization

In summary, we designed, synthesized, and characterized two novel NO-releasing phthalocyanines, namely ZnPc-2NO and ZnPc-4NO. Upon interaction with the intracellular GSH, they released NO that could inhibit the cellular respiration and downregulate the HIF-1α protein level, relieving hypoxia in HT29 and A549 cells. Upon light irradiation, both compounds exhibited high cytotoxicity under both normoxic and hypoxic conditions, demonstrating their ability for oxygen-economized PDT. The comparable photocytotoxicity of ZnPc-2NO and ZnPc-4NO suggested that two NO donors per a ZnPc core are sufficient for suppressing the cellular respiration. Interestingly, ZnPc-2NO could also trigger ICD and the release of DAMPs in HT29 cells upon photosensitization, leading to the maturation of DCs and upregulation of antitumor cytokines. In addition, the ZnPc-2NO-mediated PDT could suppress the tumor growth in 4T1 tumor-bearing mice and induce the recruitment of leukocytes and B cells into the lymph nodes and pDCs in the tumor. The overall results showed that the ZnPc-2NO-mediated PDT is an effective strategy that can overcome the tumor hypoxia and induce ICD, which can activate a robust antitumor immune response.

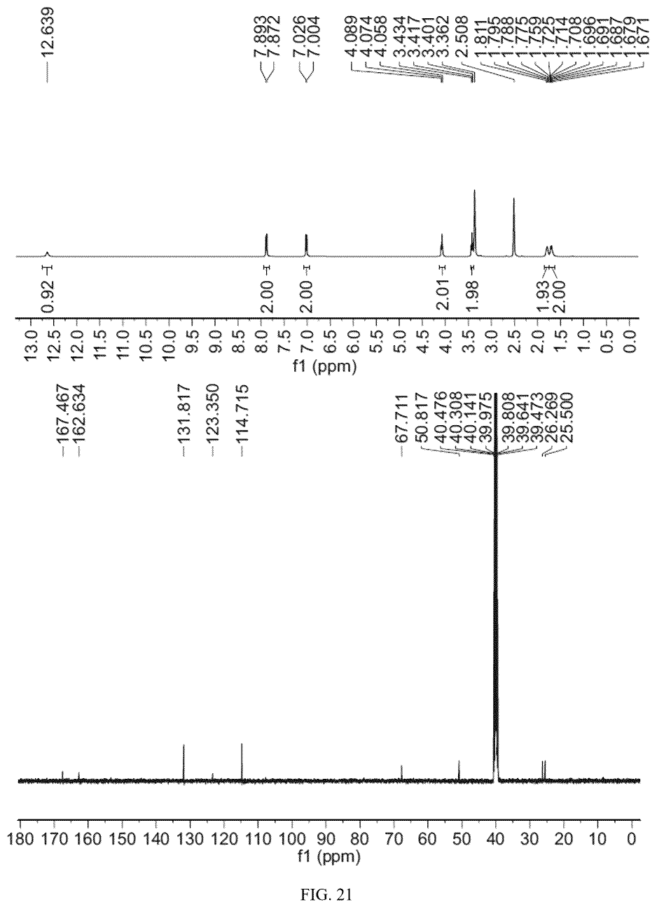

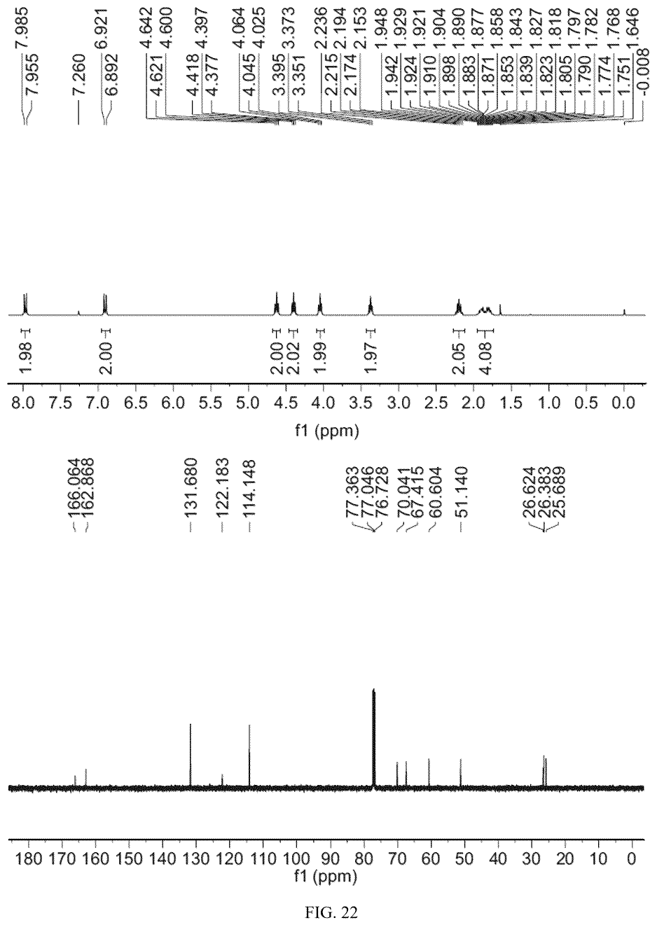

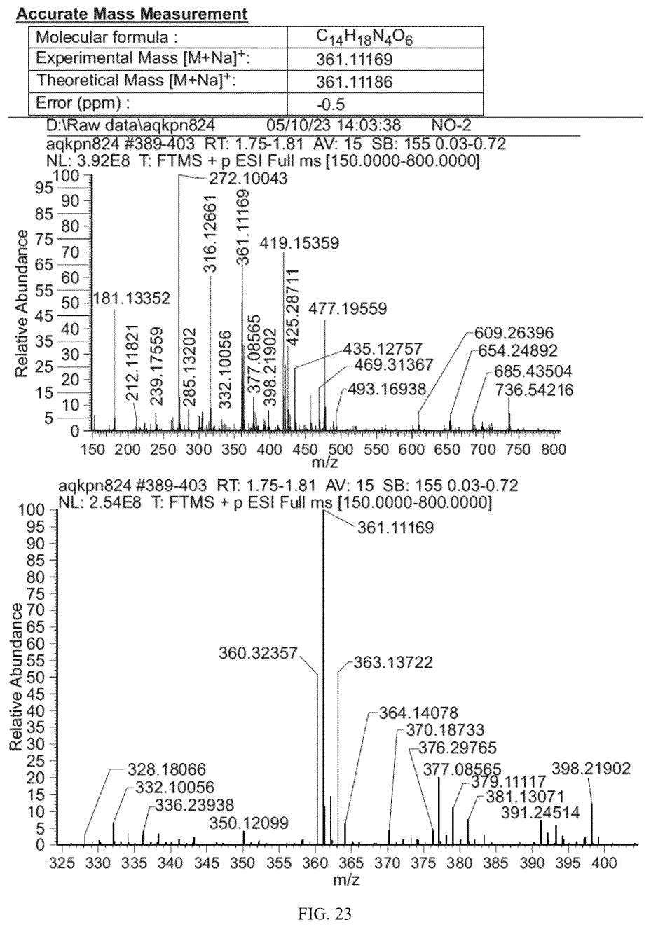

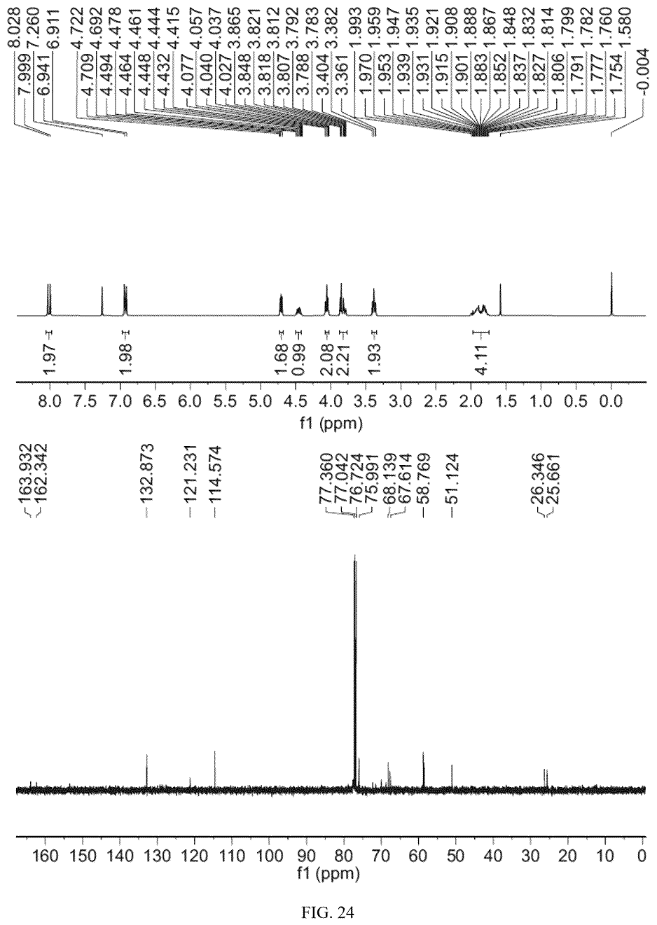

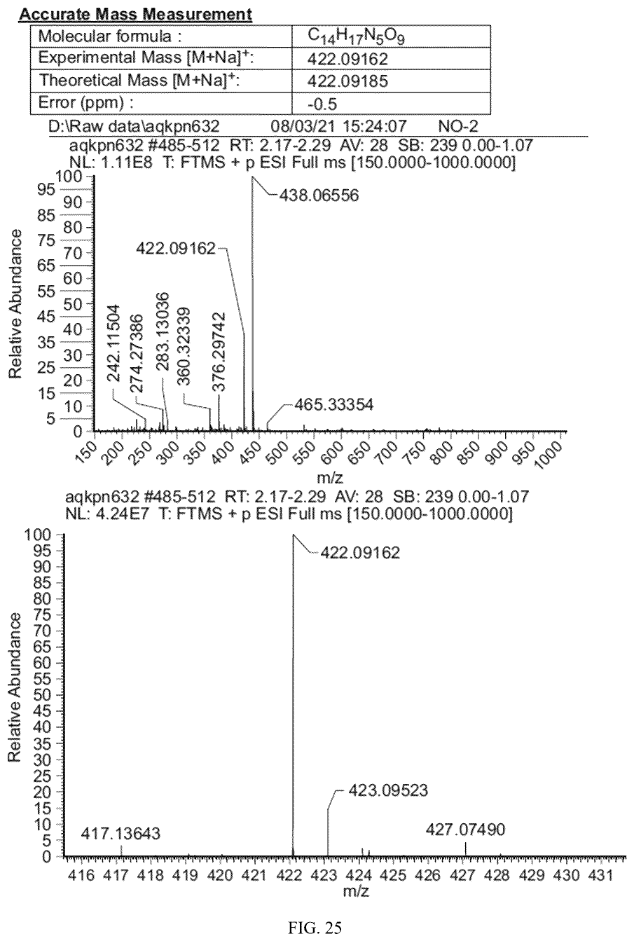

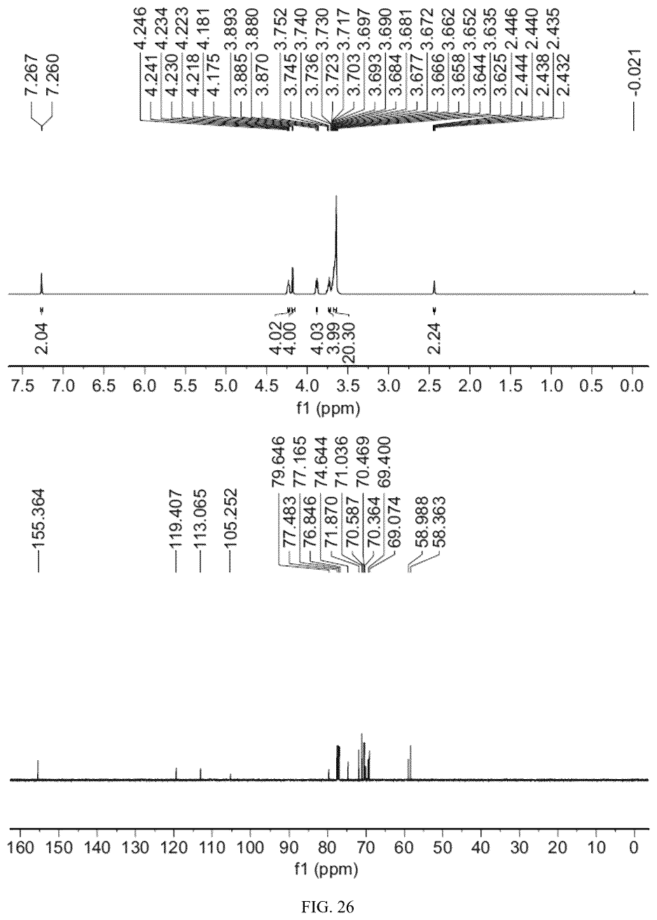

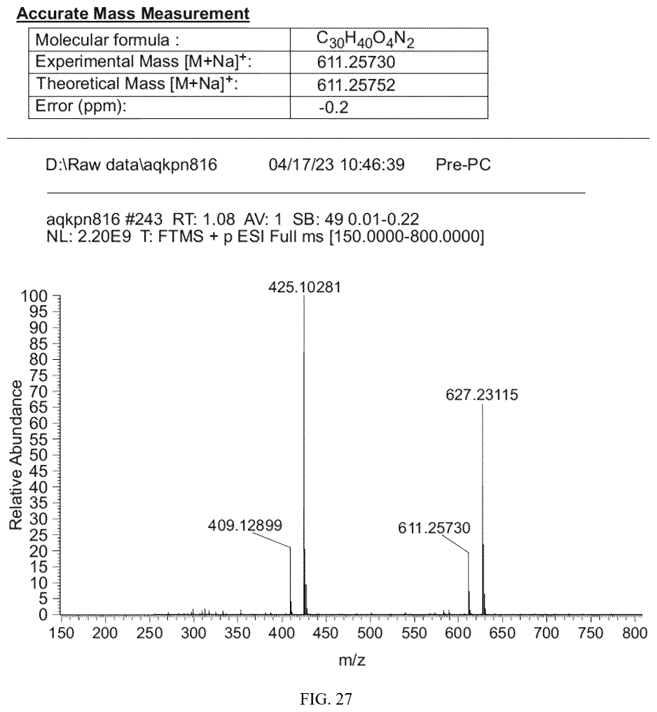

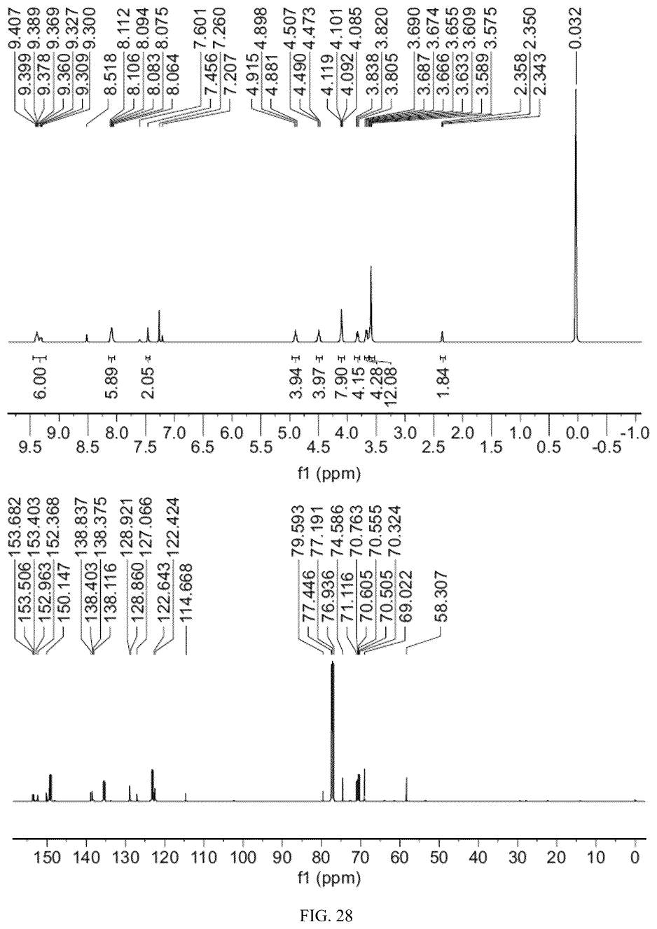

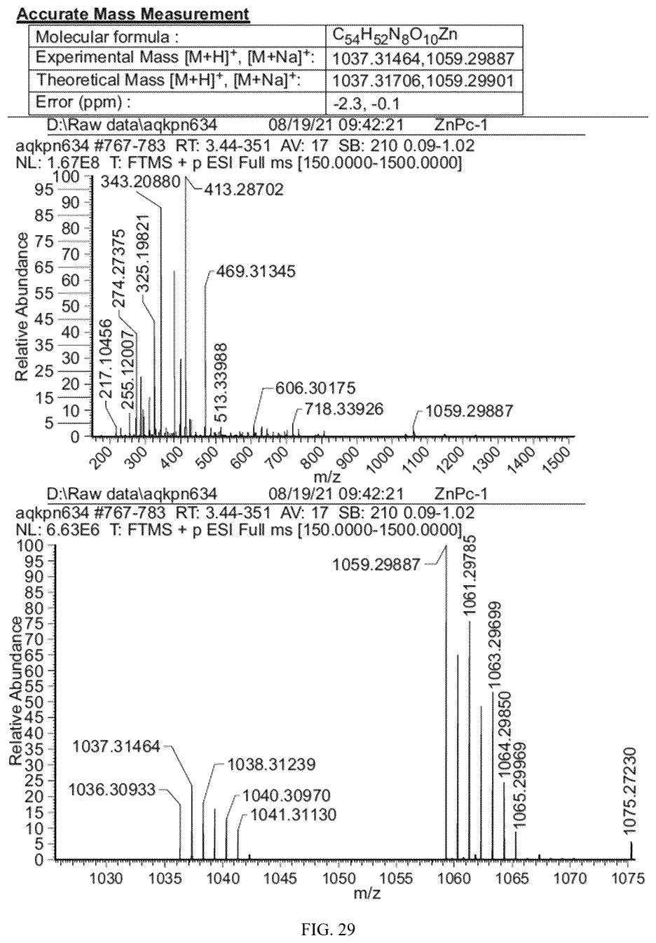

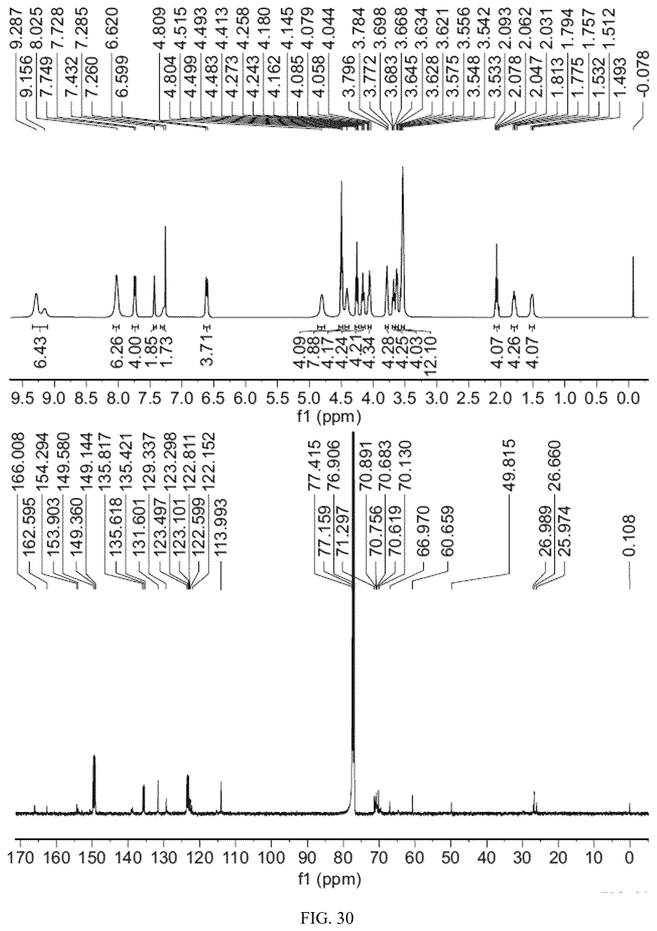

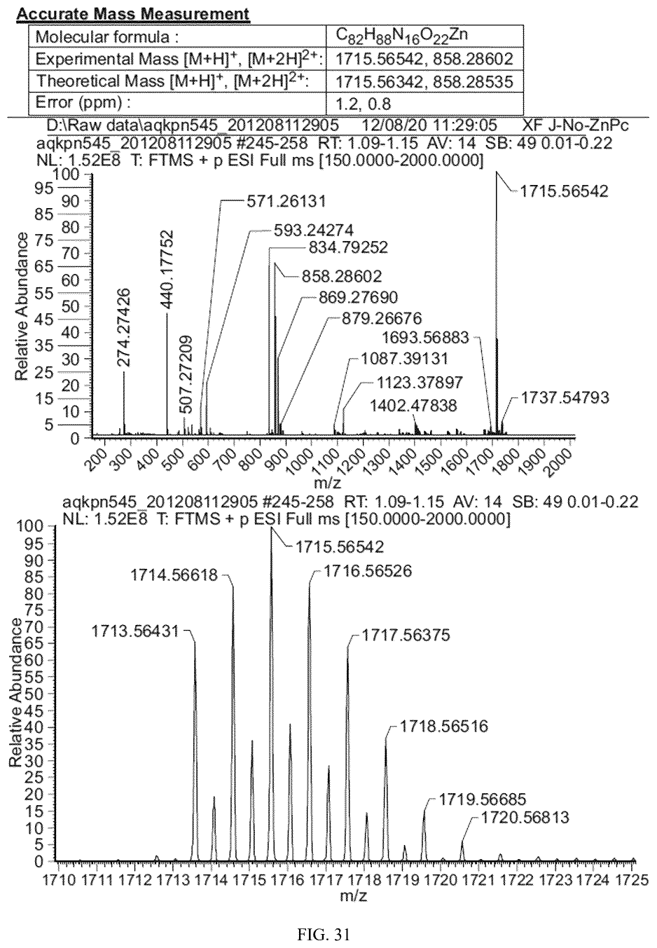

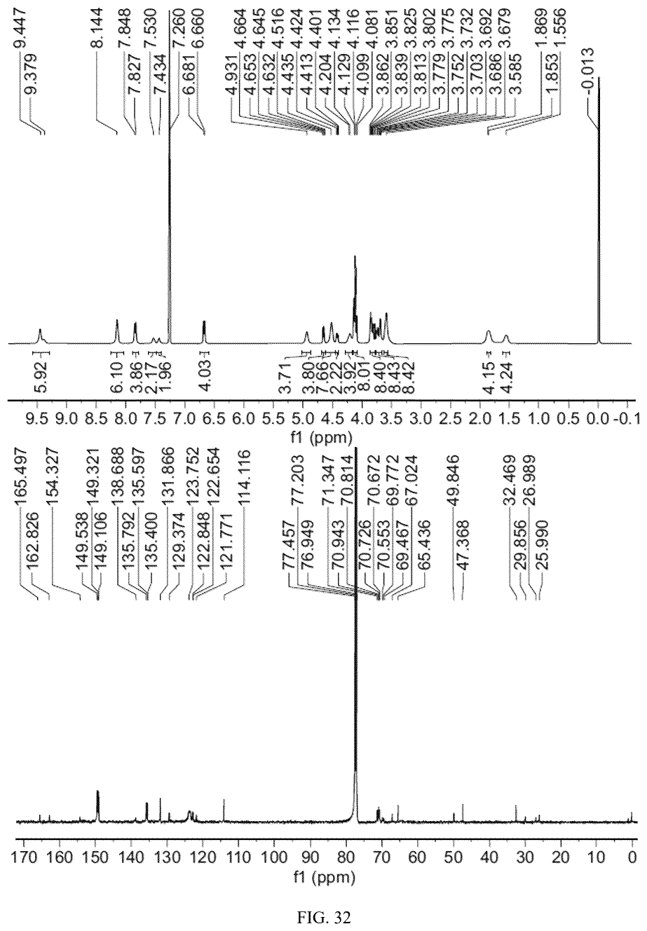

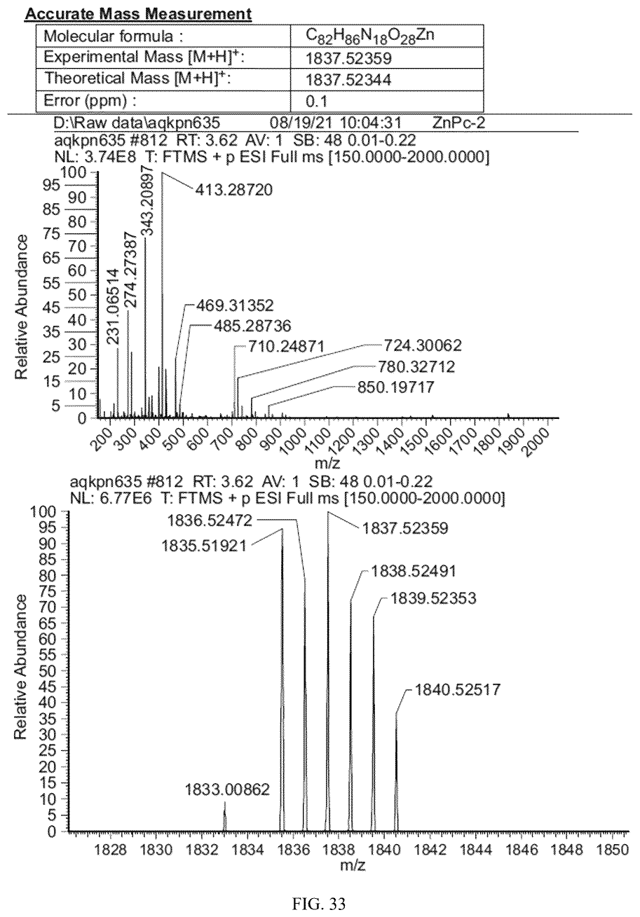

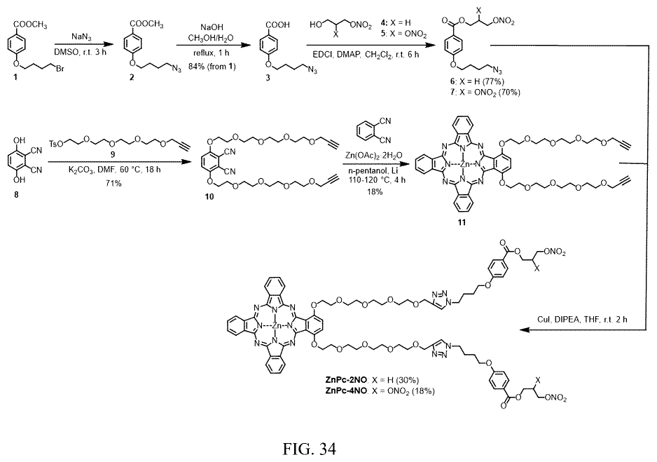

Among the different types of NO donors, organic nitrates were chosen because of their superior properties, including the rapid release of NO upon interaction with GSH, high safety profile of the by-products, and ease of preparation and chemical modification.^{[18]} For the photosensitizing unit, a ZnPc was employed owing to the strong absorption in the far-red region, balanced fluorescence emission and ROS generation, and high stability of this class of functional dyes.^{[25]} The target compounds were synthesized by a convergent approach outlined in FIG. 33. Substitution of methyl 4-(4-bromobutoxy)benzoate (1)^{[21]} with NaN₃ in dimethyl sulfoxide (DMSO) gave 2, which was then hydrolyzed to afford benzoic acid 3. This compound was then coupled with NO donor 4^{[22]} or 5^{[23]} in the presence of 1-ethyl-3-(3-dimethylaminopropyl)carbodiimide (EDCI) and 4-dimethylaminopyridine (DMAP) to give the corresponding esters 6 and 7, respectively. To prepare the ZnPc component, 2,3-dicyanohydroquinone (8) was treated with tosylate 9^{[24]} and K₂CO₃ in N,N-dimethylformamide (DMF) to give the dialkynyl phthalonitrile 10, which underwent based-promoted mixed cyclization with an excess of phthalonitrile in the presence of Zn(OAc)₂·2H₂O to give ZnPc 11, which could be isolated in 18% yield using column chromatography followed by size-exclusion chromatography. With two alkynyl moieties, this compound could couple with two units of the azido NO donors 6 and 7 via copper-catalyzed azide-alkyne cycloaddition to give the target conjugates ZnPc-2NO and ZnPc-4NO, respectively. All the new compounds were characterized by various spectroscopic methods (FIGS. 21-33).

### Example 2—Spectroscopic Properties and No Release Kinetics

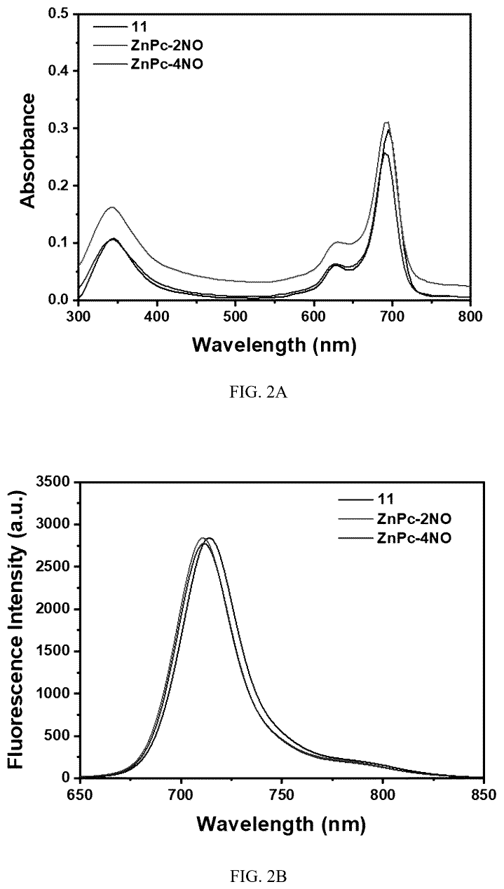

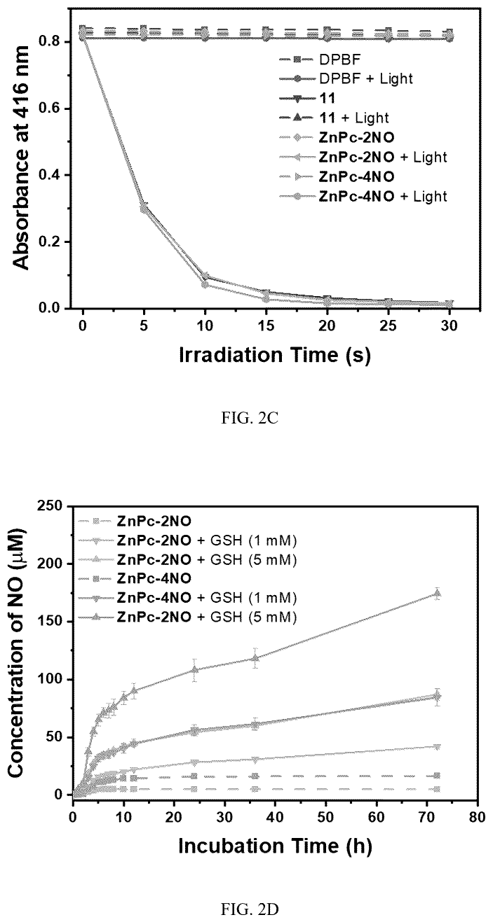

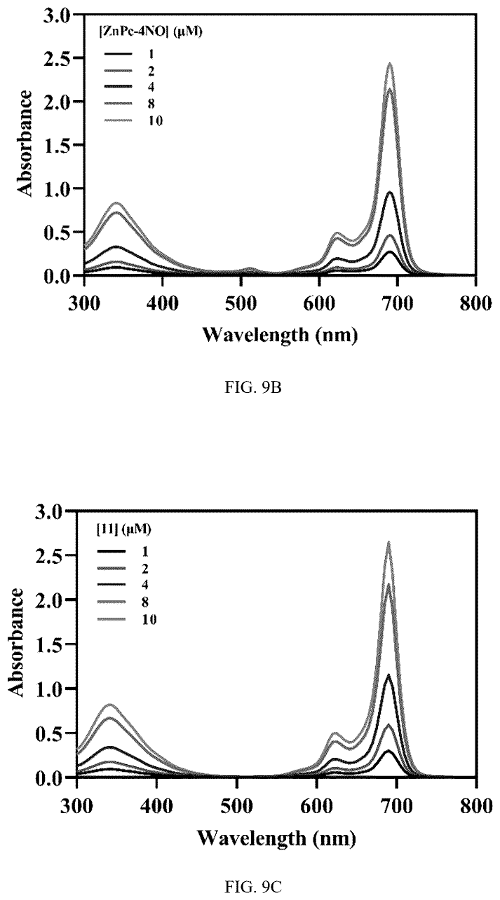

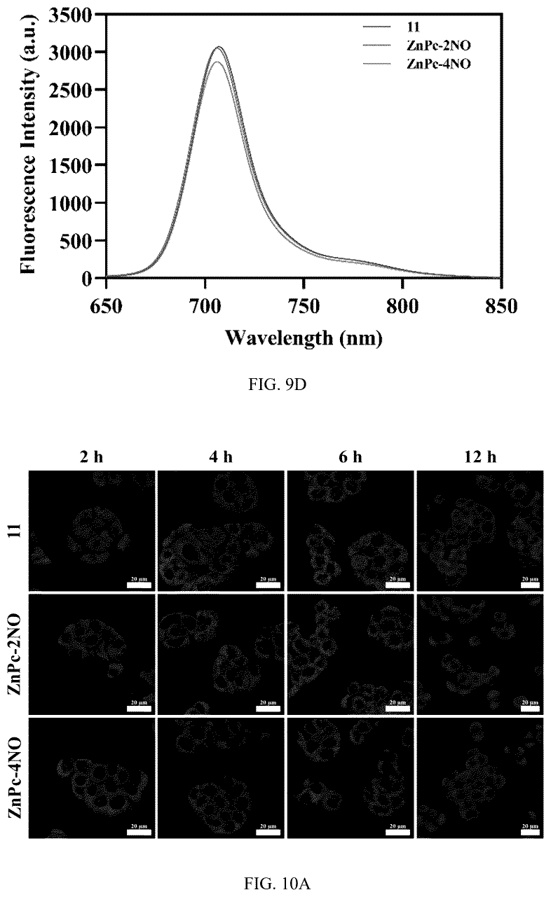

The electronic absorption and photophysical properties of ZnPc-2NO and ZnPc-4NO were first measured in DMF, using the non-NO-substituted analogue 11 for comparison. All the compounds exhibited typical electronic absorptions of zinc(II) phthalocyanines, displaying a B-band at 350 nm, a weak vibronic band at 610 nm, and a sharp and intense Q-band at 690 nm in the electronic absorption spectrum (FIGS. 9A-9C). These spectral features indicated that the compounds are essentially non-aggregated under these conditions. Upon excitation at 610 nm, these compounds showed a strong fluorescence emission at 708-710 nm with a fluorescence quantum yield (Φ_{F}) of 0.15-0.16 relative to the unsubstituted ZnPc in DMF (Φ_{F}=0.28)^{[26]} (FIG. 9D and Table 1). These spectral properties were also measured in phosphate-buffered saline (PBS) in the presence of DMF (10% v/v) and Tween 80 (1% v/v). All the compounds also exhibited a sharp and intense Q-band at 690-694 nm (FIG. 2A) and a strong fluorescence emission at 711-714 nm (FIG. 2B) in this aqueous medium. The virtually identical spectral features showed that the terminal NO-releasing units do not significantly perturb the electronic state of the ZnPc core in both of these media.

The singlet oxygen generation efficiency of these compounds was then examined in DMF using 1,3-diphenylisobenzofuran (DPBF) as the singlet oxygen scavenger. Upon light irradiation, all of them could generate singlet oxygen efficiently as reflected by the rapid decrease in the absorbance at the DPBF's absorption at 416 nm (FIG. 2C). The singlet oxygen quantum yields (Φ_{Δ}) were determined to be 0.60-0.64 relative to the unsubstituted ZnPc (Φ_{Δ}=0.56)^{[26]} (Table 1). Table 1 compiles all these spectroscopic and photophysical data recorded in DMF, including those of the unsubstituted ZnPc for comparison.

TABLE 1
Electronic absorption and photophysical
data of ZnPc-2NO, ZnPc-4NO, and 11 in DMF

| | | λₑₘ | | |
|---|---|---|---|---|
| Compound | λ_{abs} (nm) (log ε) | (nm)^{[a]} | Φ_{F}^{[b]} | Φ_{Δ}^{[c]} |
| ZnPc-2NO | 350 (4.96), 610 (4.54), 690 (5.50) | 710 | 0.16 | 0.60 |
| ZnPc-4NO | 350 (4.93), 610 (4.51), 690 (5.43) | 708 | 0.16 | 0.61 |
| 11 | 350 (4.94), 610 (4.56), 690 (5.48) | 708 | 0.15 | 0.64 |
| ZnPc | 350 (4.52), 610 (4.27), 670 (5.15) | 685 | 0.28 | 0.56 |

^{[a]}Excited at 610 nm.
^{[b]}Relative to ZnPc (Φ_{F} = 0.28 in DMF).
^{[c]}Relative to ZnPc (Φ_{Δ} = 0.56 in DMF).

The GSH-responsive release of NO from ZnPc-2NO and ZnPc-4NO was then monitored using a Griess assay.^{[27]} In this assay, the released NO is first oxidized rapidly to nitrite (NO₂⁻), which is a stable and non-volatile metabolite of NO, and this species further reacts with sulphanilamide (Griess reagent I) and then N-(1-naphthyl)ethylenediamine (Griess reagent II) to form a diazonium species with an absorption at 540 nm. The NO concentration can be determined by measuring the absorbance at this position with reference to a standard curve of NaNO₂ aqueous solution. To perform this study, solutions of ZnPc-2NO and ZnPc-4NO in DMF (250 μM) with or without GSH (1 or 5 mM) were prepared. At different time points, an aliquot of the mixtures (50 μL) was withdrawn, and the amount of NO released was determined by this colorimetric NO assay. As shown in FIG. 2D, while both conjugates could not generate NO in the absence of GSH, the addition of GSH could promote the release of NO, and both the extent and rate of release were higher when a higher concentration of GSH was used. With two additional nitrate substituents, ZnPc-4NO could generate more NO as expected.

### Example 3—In Vitro Studies

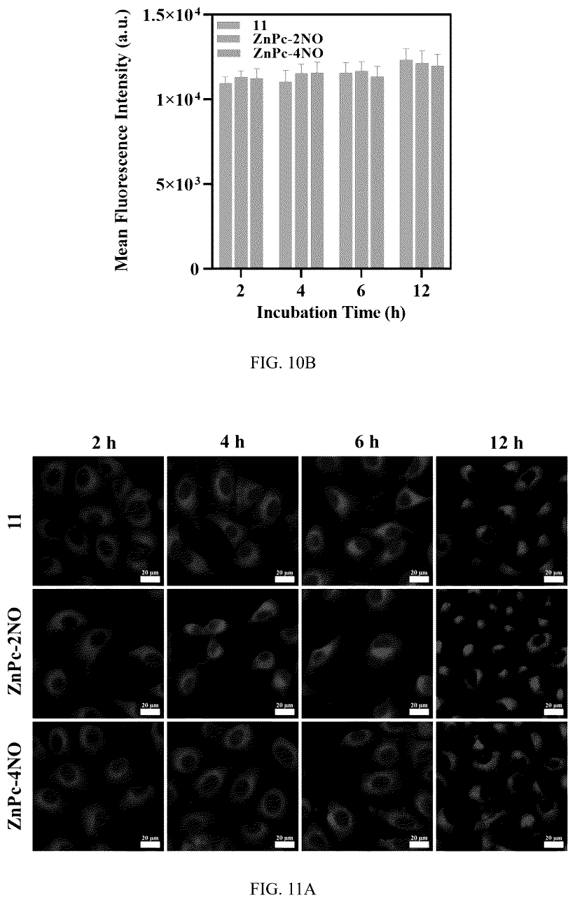

The cellular uptake of 11, ZnPc-2NO, and ZnPc-4NO was then studied against HT29 human colorectal adenocarcinoma cells using confocal microscopy and flow cytometry. It was found that all of them could be internalized readily, exhibiting a strong fluorescence signal inside the cells, and the quantified intracellular fluorescence intensities were not significantly different for the three compounds and for the four incubation times used (2, 4, 6, and 12 h) (FIGS. 10A and 10B).

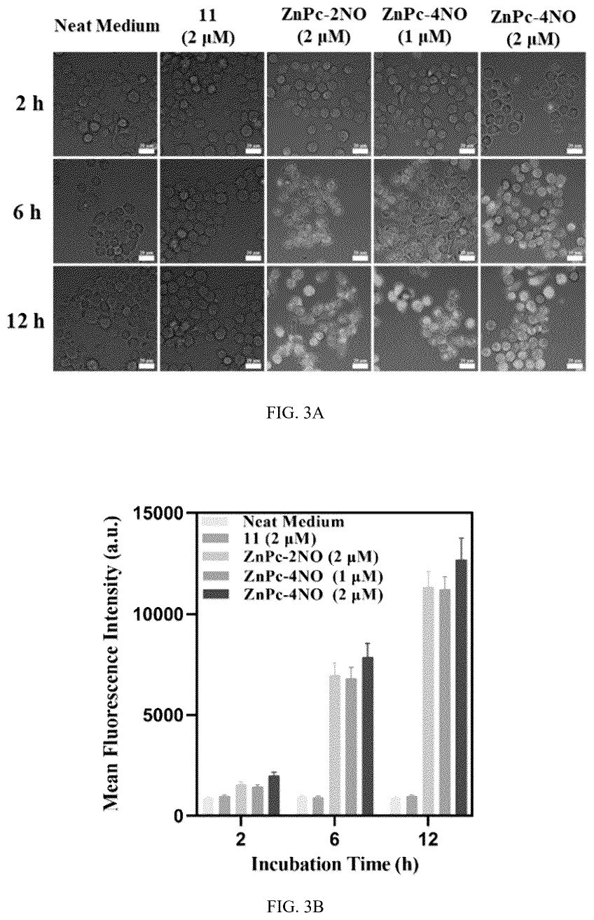

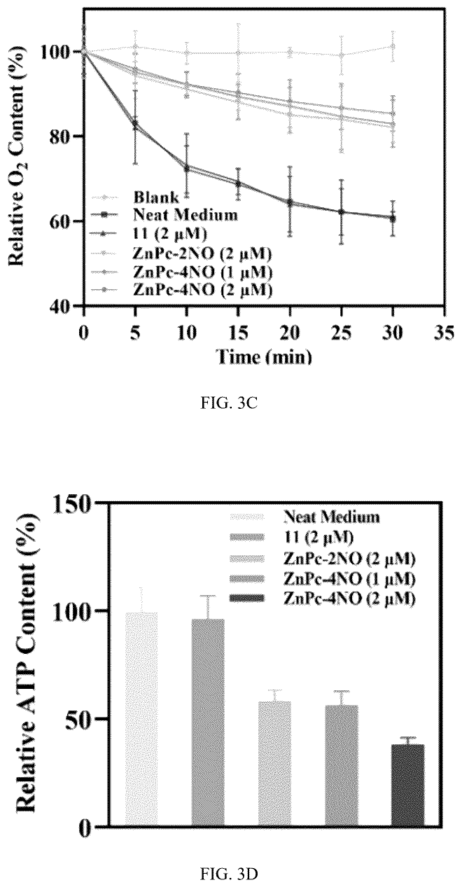

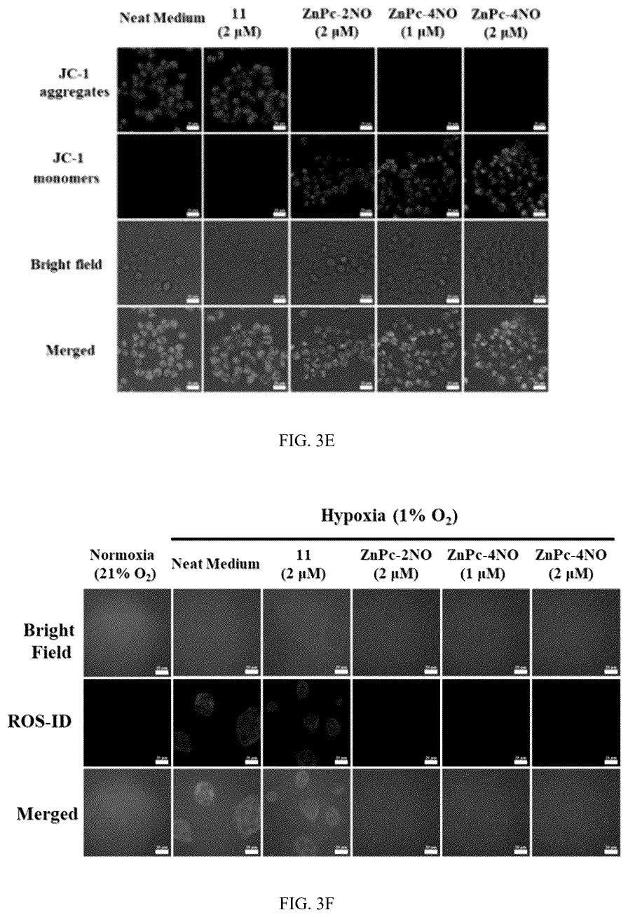

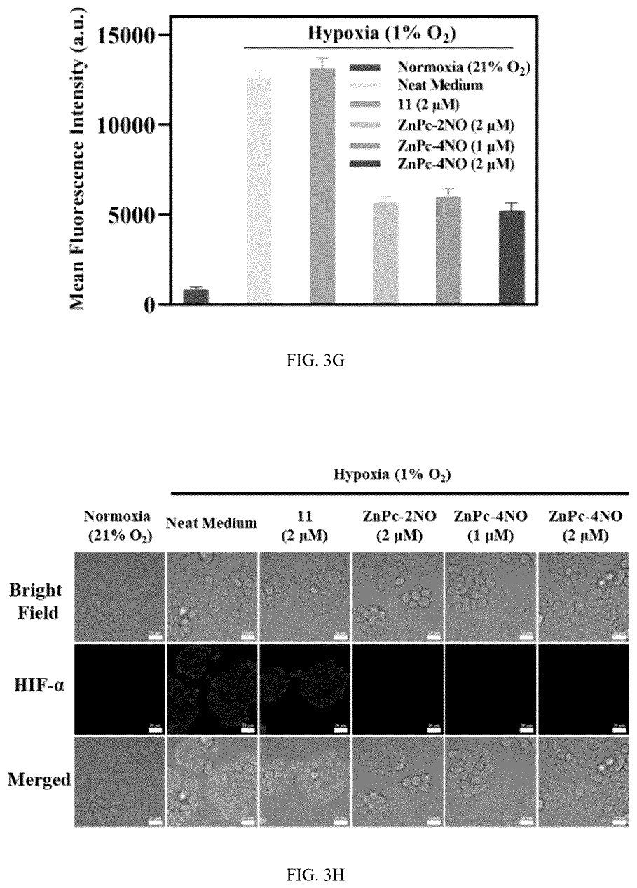

The release of NO from ZnPc-2NO and ZnPc-4NO inside the cells was then studied using 4-amino-5-methylamino-2,7-difluorofluorescein diacetate (DAF-FM-DA) as the probe,^{[28]} which is converted into 4-amino-5-methylamino-2,7-difluorofluorescein (DAF-FM) upon hydrolysis by the intracellular esterase, and then it reacts with NO to form a highly fluorescent benzotriazole fluorescein derivative (DAF-FM-T). Briefly, HT29 cells were incubated in the culture medium with or without 11, ZnPc-2NO, or ZnPc-4NO (1 or 2 μM) for different periods of time (2, 6, or 12 h), followed by the incubation with DAF-FM-DA (5 μM) for 30 min. The cellular images and the intracellular fluorescence intensities were then determined and compared. As shown in FIG. 3A, the signal of DAF-FM-T could clearly be seen for the cells being treated with ZnPc-2NO or ZnPc-4NO, but not for those being incubated in the culture medium with or without 11. The intensity was increased with the incubation time, showing that NO was released from these cells in a time-dependent manner. The mean intracellular fluorescence intensity for ZnPc-2NO (at 2 μM) was very similar to that for ZnPc-4NO (at 1 μM), which has 2-fold NO-releasing units compared with the former (FIG. 3B). Surprisingly, when 2 μM of ZnPc-4NO was used, the intensity was only marginally increased, which was not consistent with the results in solution study (FIG. 2D). It might be attributed to the insufficient GSH inside the cells to trigger the release of NO or the limited sensitivity of the NO probe.

The effect of these conjugates on the cellular respiration was also investigated by monitoring the relative oxygen content in the medium containing the cancer cells over a period of time. In this study, HT29 cells were incubated in the culture medium with or without 11, ZnPc-2NO, or ZnPc-4NO (1 or 2 μM) for 8 h, during which the medium was sealed with liquid paraffin to exclude external oxygen supply. The dissolved oxygen content in the medium was measured by an oxygen meter at different time points over a further period of 30 min. As shown in FIG. 3C, the oxygen content was largely decreased (to ca. 60% in 30 min) in the medium containing the cells with or without the treatment with 11, which could be attributed to regular cellular respiration. Upon treatment with ZnPc-2NO or ZnPc-4NO, the oxygen content was significantly higher and decreased in less than 20% in 30 min. Considering that ATP is the main product of cellular respiration, the intracellular ATP content was also determined after the aforementioned treatments using a luminescent ATP detection kit. As shown in FIG. 3D, the ATP content was reduced to about 50% after the treatment with ZnPc-2NO (2 μM) or ZnPc-4NO (1 μM) for 12 h compared with those in the cells being incubated in the medium with or without 11. The reduction was further enhanced when the concentration of ZnPc-4NO was increased to 2 μM. All these results suggested that the cellular respiration of these cancer cells could be inhibited by these conjugates due to the released NO.

In addition, the influence of the conjugates on the mitochondrial membrane potential (ΔΨₘ) was also investigated using 5,5′,6,6′-tetrachloro-1,1′,3,3′-tetraethylbenzimidazolo-carbocyanine iodide (JC-1), which exhibits potential-dependent accumulation in mitochondria. At high ΔΨₘ, JC-1 forms aggregates and exhibits red fluorescence at 590 nm, while it exists as a monomer at low ΔΨₘ and emits green fluorescence at 525 nm. The decrease in the ratio of red to green fluorescence intensity indicates mitochondrial depolarization, which is a feature of mitochondrial dysfunction. To perform this study, HT29 cells were incubated in the culture medium with or without 11, ZnPc-2NO, or ZnPc-4NO (1 or 2 μM) for 12 h, followed by staining with JC-1 (10 μg mL⁻¹) for 30 min. It was found that the mitochondrial membrane was depolarized in the cells being treated with ZnPc-2NO or ZnPc-4NO, while minimal changes were observed for the 11-treated cells (FIG. 3E), indicating that the released NO also induced mitochondrial dysfunction.

The hypoxic status of the cells after these treatments under a hypoxic condition (with 1% oxygen) was further examined using the ROS-ID fluorogenic hypoxia probe, in which the nitro group is converted to hydroxylamine and an amine by the nitroreductase inside the hypoxic cells to release a fluorophore. As shown in FIG. 3F, a strong fluorescence signal was observed for HT29 cells being cultured in a neat medium or in the presence of 11. For the cells being incubated with ZnPc-2NO or ZnPc-4NO, the intracellular fluorescence was significantly weaker and comparable with that for the cells being cultured under a normoxic (with 21% oxygen) condition. The fluorescence intensities under these conditions were also quantified by flow cytometry. It was found that the intensity was reduced by ca. 60% after the treatment with ZnPc-2NO or ZnPc-4NO and increasing the concentration of ZnPc-4NO did not lead to further reduction of the intensity (FIG. 3G). This again indicated that even though ZnPc-4NO could release more NO in solution, it was not the case inside the cells, and ZnPc-2NO was already an effective NO-releasing photosensitizer.

As HIF-1α is usually upregulated in tumors and plays an important role in tumor metastasis and angiogenesis,^{[5]} its expression in the HT29 cells being treated as described above was also examined using an immunostaining method. The trend of the HIF-1α level, as reflected by the relative intracellular fluorescence intensity (FIGS. 3H and 3I), was in good agreement with that of the hypoxic status of the cells (FIGS. 3F and 3G), confirming that these NO-releasing compounds could mitigate hypoxia and downregulate HIF-1α by inhibiting the cellular respiration.

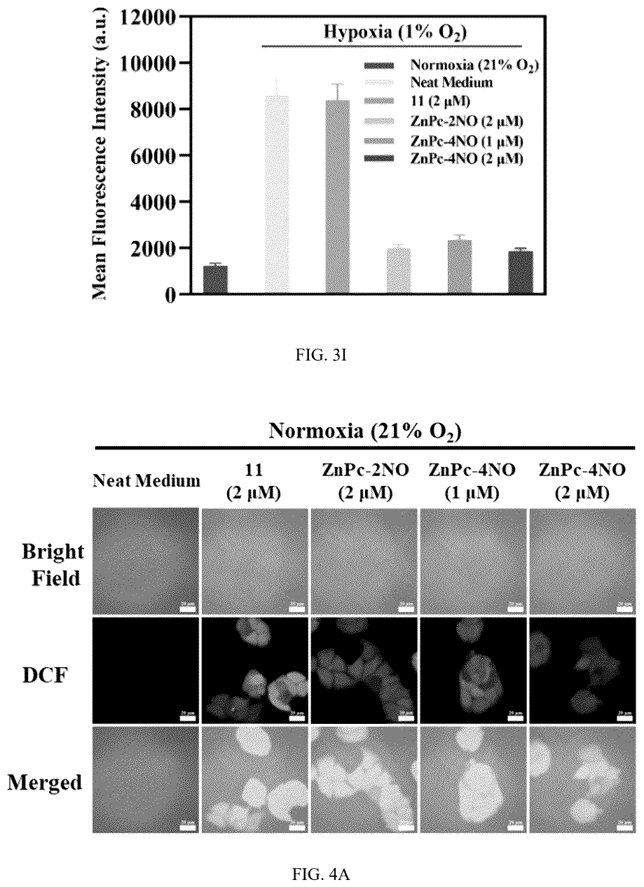

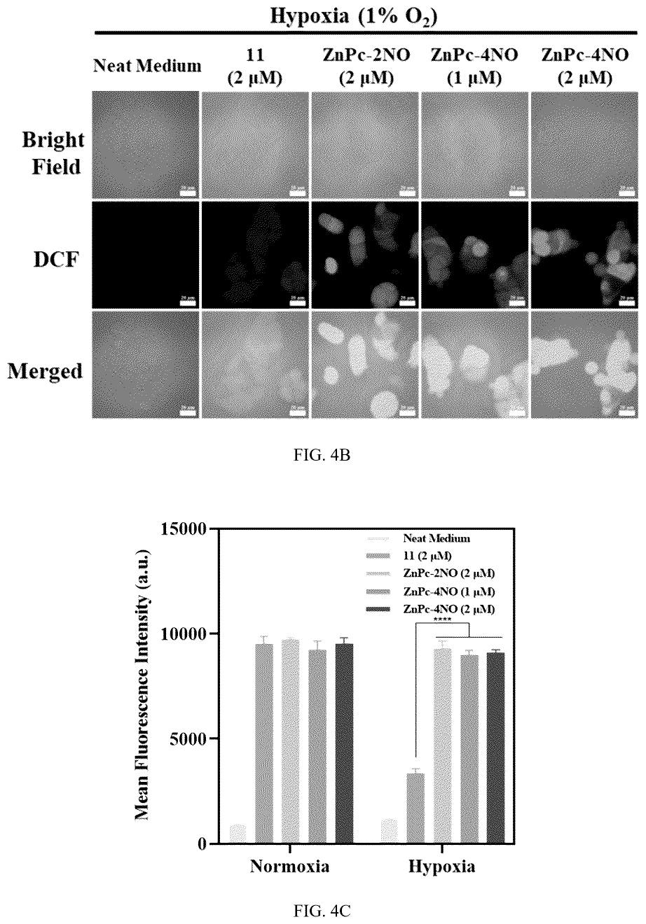

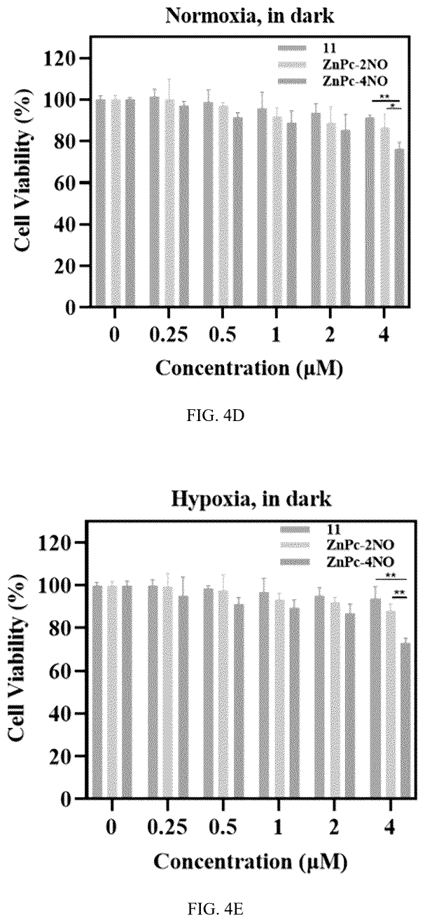

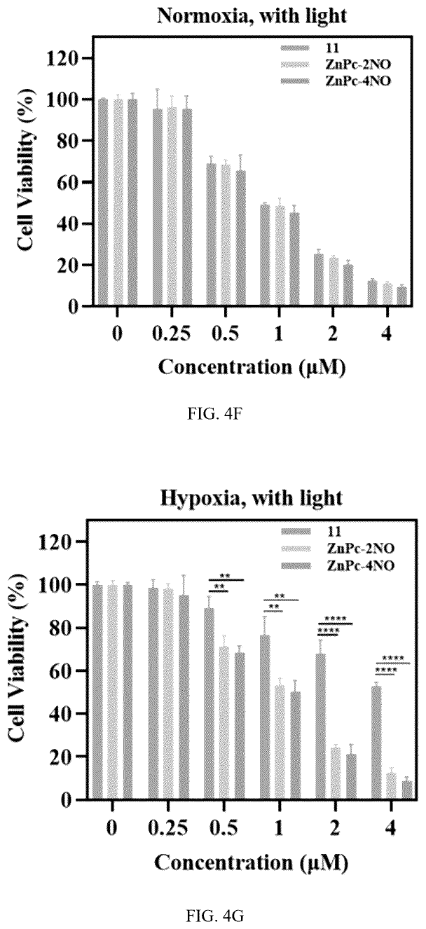

The NO-inhibited cellular respiration was believed to conserve more intracellular oxygen for the photodynamic action, which might overcome the problem of tumor hypoxia. To validate this hypothesis, the ROS generation inside the HT29 cells after the aforementioned treatments was examined under both normoxic and hypoxic conditions, using 2′,7′-dichlorodihydrofluorescein diacetate (H₂DCFDA) as a probe, which would be deacetylated by the esterase and then oxidized by the ROS inside the cells to give highly fluorescent 2′,7′-dichlorofluorescein (DCF). It was found that all the compounds could generate ROS upon light irradiation under a normoxic condition, as shown by the strong fluorescence of DCF (FIG. 4A). In contrast, only ZnPc-2NO and ZnPc-4NO induced ROS inside the cells under hypoxia, whereas the ROS generation was largely inhibited in the cells being incubated with 11 (FIG. 4B). For the 11-treated HT29 cells, the quantified intracellular fluorescence intensity of DCF was only about 30% of that for the ZnPc-2NO and ZnPc-4NO-treated counterparts (FIG. 4C).

The dark and photo-cytotoxicities of ZnPc-2NO and ZnPc-4NO were further investigated under both normoxic and hypoxic conditions and compared with those of 11. In the absence of light irradiation, all the photosensitizers were found to be essentially non-cytotoxic up to a concentration of 1-2 μM. At 4 μM, ZnPc-4NO became slightly cytotoxic, reducing the cell viability to ca. 30%. The results were similar under normoxic (FIG. 4D) and hypoxic (FIG. 4E) conditions. Upon light irradiation (λ>610 nm, 18 mW cm⁻², 1.08 J cm⁻²), all the photosensitizers showed comparable and concentration-dependent cytotoxicity under normoxia (FIG. 4F). Interestingly, while the photocytotoxicity of 11 was significantly reduced under hypoxia, ZnPc-2NO and ZnPc-4NO remained highly potent and could kill almost all the cells at a drug dose of 4 μM (FIG. 4G). The overall results showed that both ZnPc-2NO and ZnPc-4NO could alleviate the hypoxic status of the cells via inhibition of the cellular respiration, thereby sparing more oxygen for effective photodynamic elimination of cancer cells even under hypoxia.

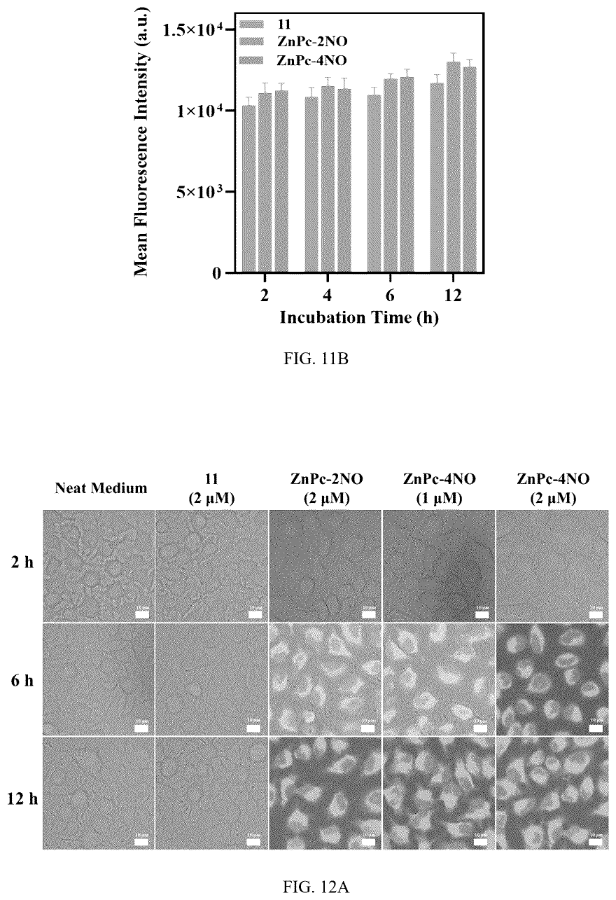

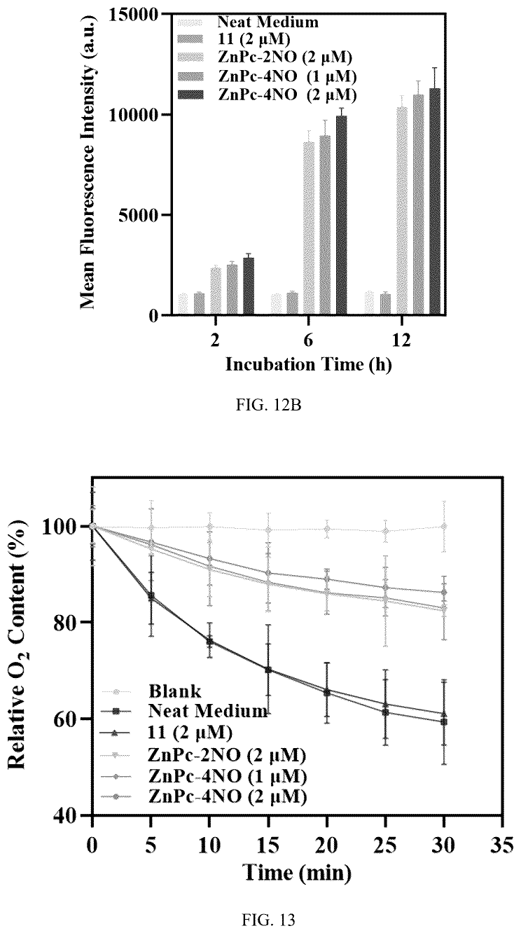

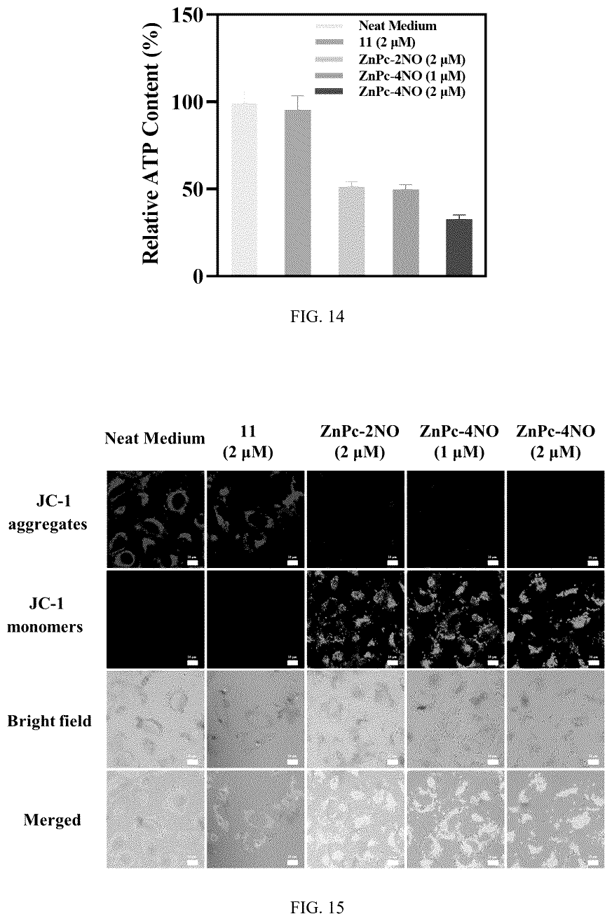

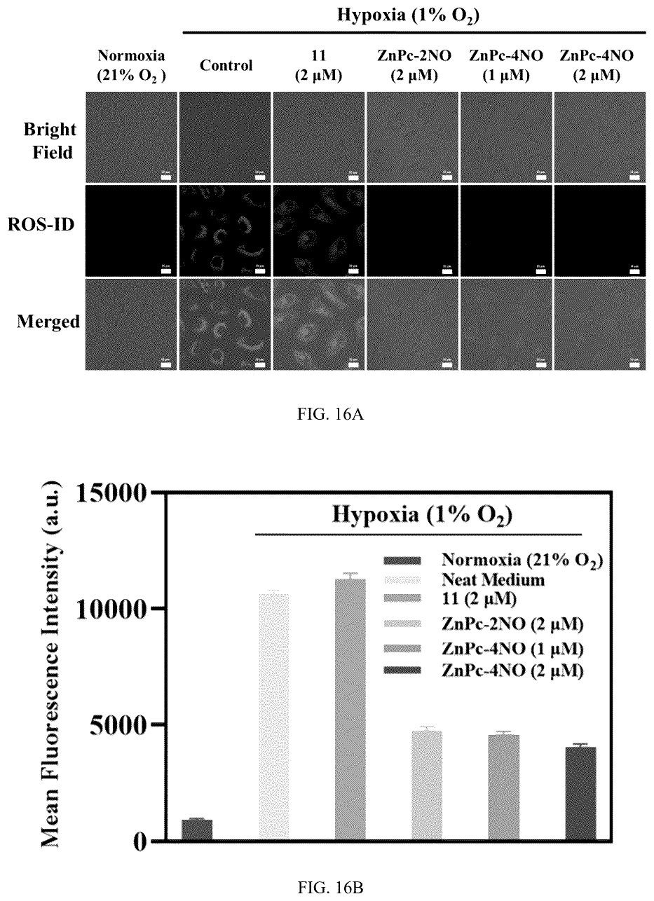

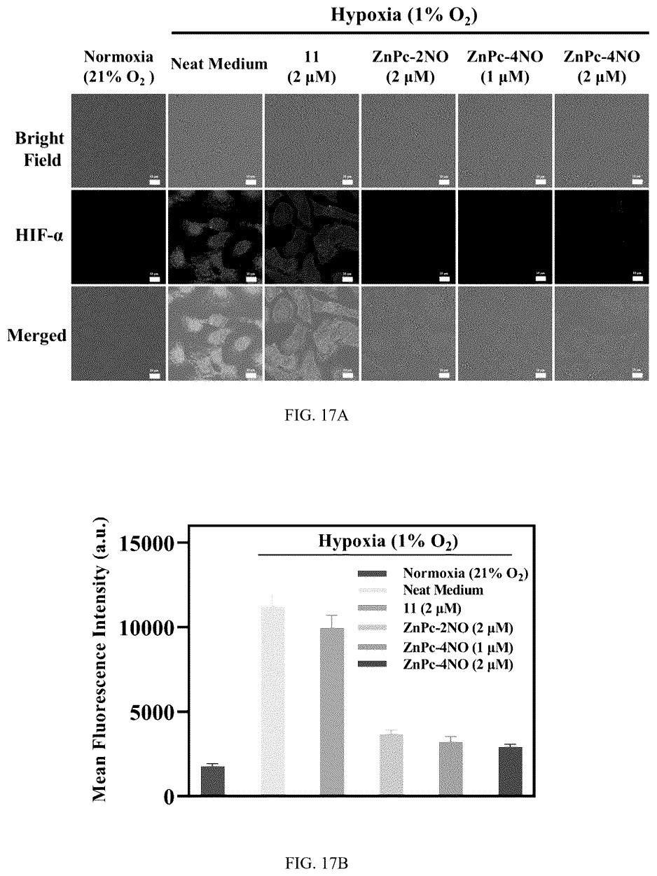

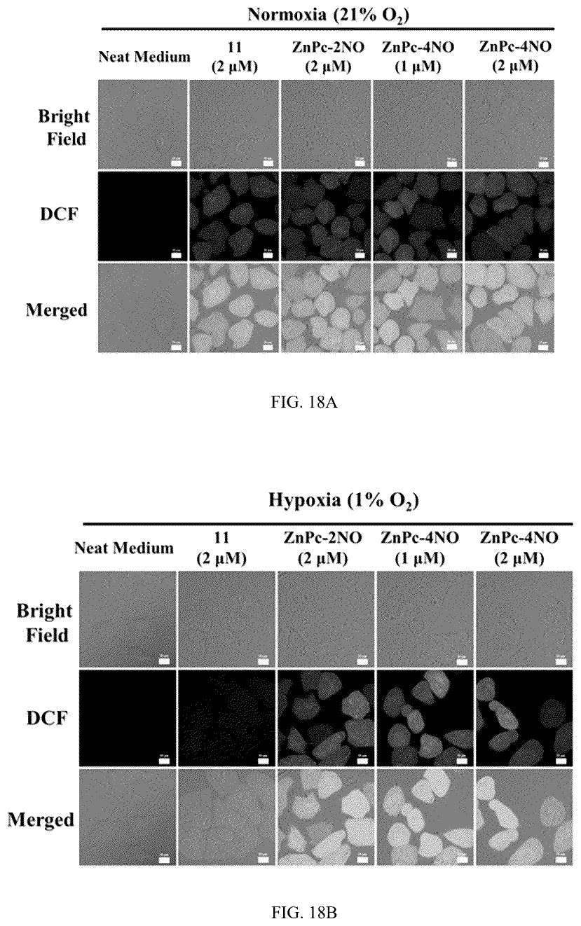

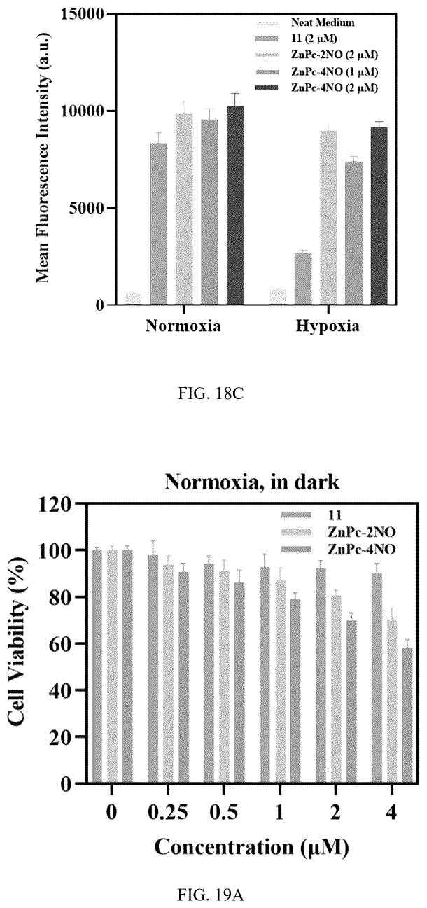

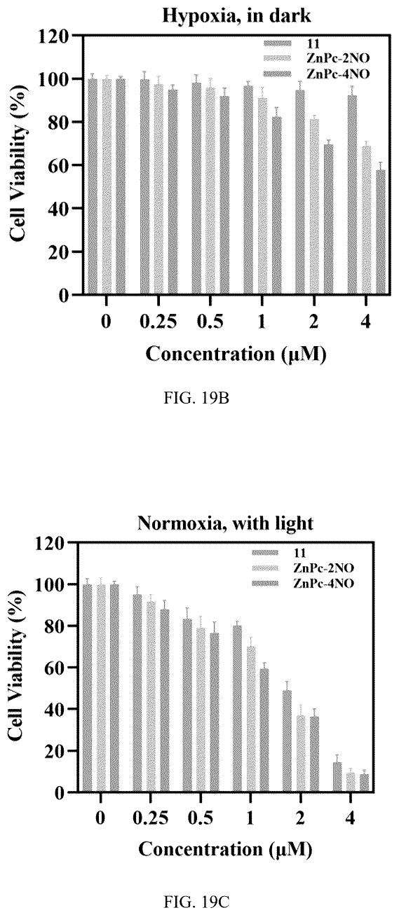

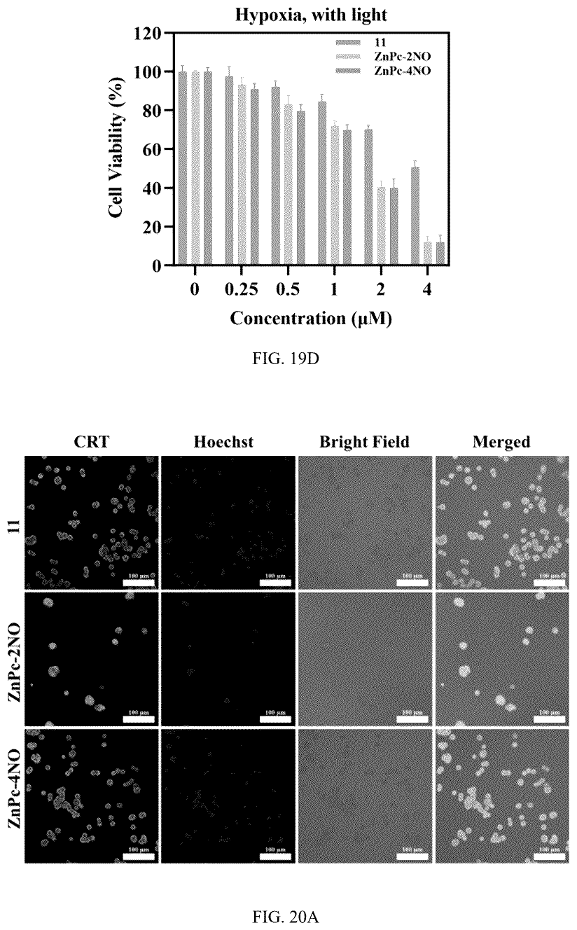

All the aforementioned in vitro studies were also performed using A549 human lung carcinoma cells. The results were very similar to those for HT29 cells (FIGS. 11A-19D) showing that the in vitro properties of ZnPc-2NO and ZnPc-4NO were not cell selective. Table 2 summarizes the half-maximal inhibitory concentrations (IC₅₀ values) of all these photosensitizers against the two cell lines under normoxic and hypoxic conditions. It can be seen that all the values for ZnPc-2NO and ZnPc-4NO were similar and in the range of 1.4-2.0 μM. In addition, while the photocytotoxicity of 11 was similar to that of ZnPc-2NO and ZnPc-4NO under normoxia, this non-NO-substituted analogue was significantly less photocytotoxic under hypoxia, and its IC₅₀ values could not be determined.

TABLE 2
IC₅₀ values of 11, ZnPc-2NO, and ZnPc-4NO against HT29
and A549 cells under normoxia and hypoxia with light irradiation
(λ > 610 nm, 18 mW cm⁻², 1.08 J cm⁻²)

| | IC₅₀ (μM) | | | |
|---|---|---|---|---|
| | Normoxia | | Hypoxia | |
| | HT29 cells | A549 cells | HT29 cells | A549 cells |
| 11 | 1.61 ± 0.06 | 2.23 ± 0.05 | —^{[a]} | —^{[a]} |
| ZnPc-2NO | 1.52 ± 0.05 | 1.92 ± 0.03 | 1.59 ± 0.05 | 2.03 ± 0.11 |
| ZnPc-4NO | 1.39 ± 0.02 | 1.73 ± 0.08 | 1.52 ± 0.09 | 1.87 ± 0.10 |

^{[a]}It could not be determined up to 4 μM.

### Example 4—Study of Immunogenic Cell Death

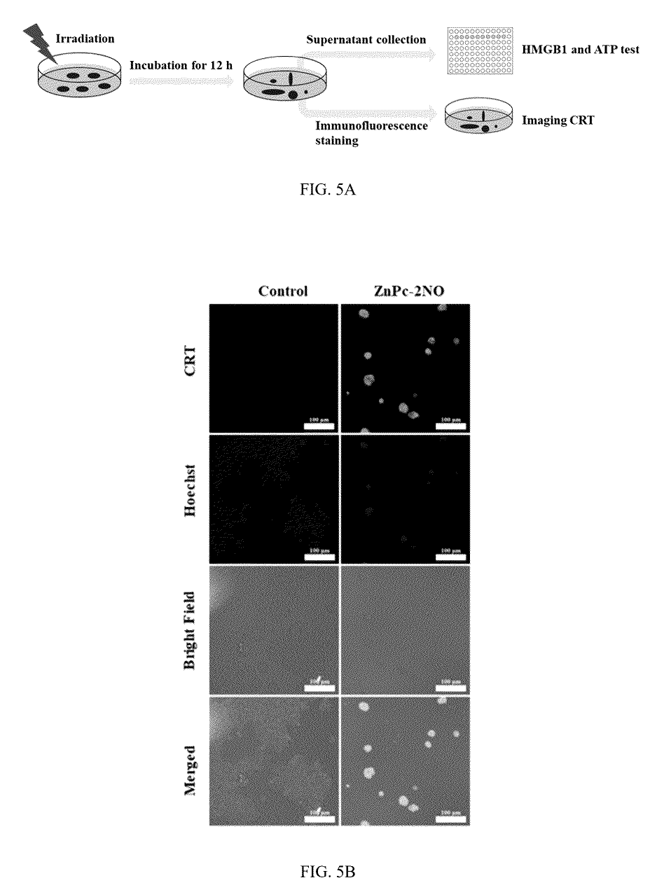

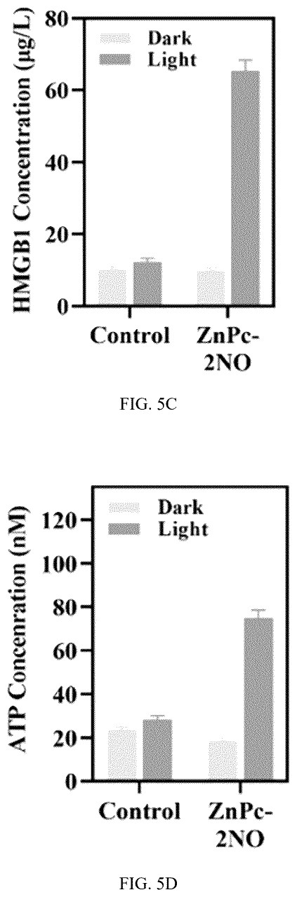

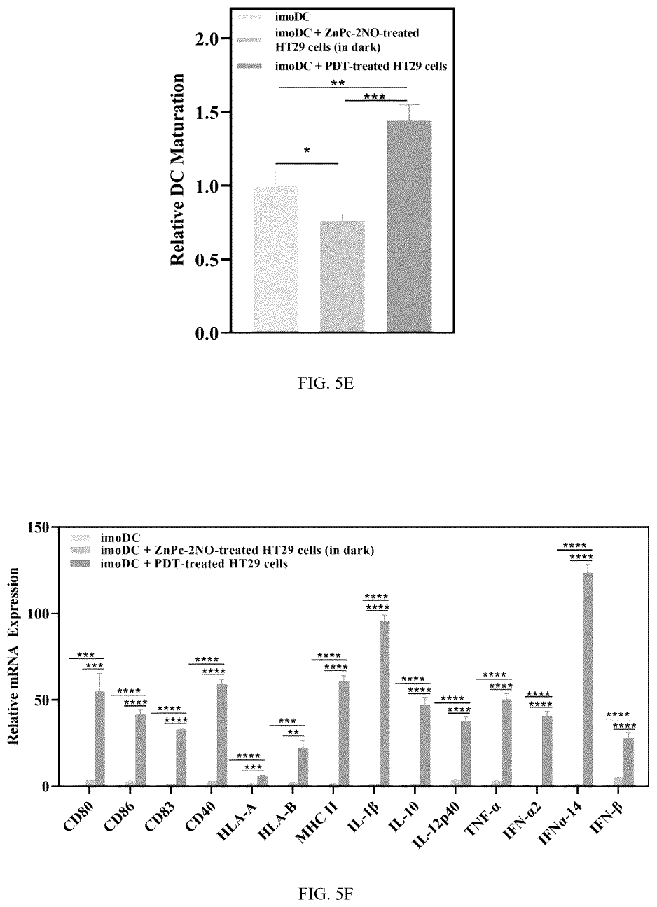

ICD is a cell death mechanism characterized by the release of various DAMPs that trigger an immune response. Previous studies have demonstrated that PDT can trigger an ICD-mediated antitumor immune response by inducing the release of tumor-associated DAMPs, such as CRT translocated from the endoplasmic reticulum to the cell surface, HMGB1 released from the nucleus, and ATP from the mitochondria, thus stimulating the maturation of DCs.^{[29]} To investigate the potential of these NO-releasing photosensitizers to induce ICD through PDT, ZnPc-2NO was selected for this study against HT29 cells. We first examined the level of cell surface CRT and the extracellular levels of HMGB1 and ATP in the cell culture medium according to the protocols outlined in FIG. 5A. We observed strong CRT immunofluorescence on the cells treated with ZnPc-2NO and light irradiation, while the signal for the cells not incubated with ZnPc-2NO was negligible (FIG. 5B). Using a colorimetric enzyme-linked immunosorbent assay (ELISA) and a luminescent ATP detection assay kit, it was found that the photodynamic action of ZnPc-2NO could significantly enhance the release of HMGB1 and ATP from the cells, as compared to the non-ZnPc-2NO-treated or non-irradiated controls (FIGS. 5C and 5D). Together, these results demonstrated that PDT sensitized by ZnPc-2NO could induce ICD and trigger the release of these DAMPs in HT29 cells.

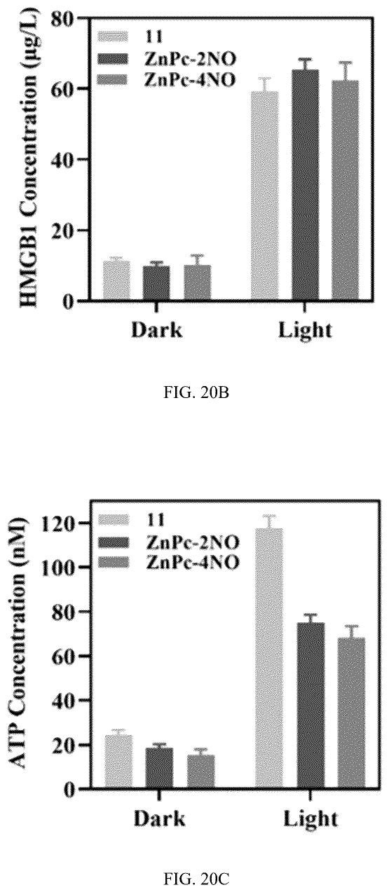

The study was then extended to ZnPc-4NO and 11 to reveal whether this effect is dependent on the substituents. It was found that these DAMPs could also be released upon the action of these photosensitizers in the presence of light irradiation, but not in dark (FIGS. 20A-20C). The extent of CRT translocation to those cell membrane (FIG. 20A) and the secreted level of HMGB1 (FIG. 20B) triggered by the photodynamic effect of these two photosensitizers were comparable with those for ZnPc-2NO. However, the ATP concentration was significantly higher in the 11-treated cell supernatant, which could be attributed to the reduced ATP levels in ZnPc-2NO and ZnPc-4NO-treated HT29 cells as a result of the NO-mediated inhibition of ATP production (FIG. 20C). These results suggested that all these zinc(II) phthalocyanines could trigger ICD upon light irradiation regardless of the presence and number of the NO donor. Nevertheless, the unique NO-releasing property of ZnPc-2NO and ZnPc-4NO could effectively retard cellular respiration and relieve the hypoxic status of cancer cells, thereby overcoming the oxygen-dependent nature of PDT. As the intracellular NO-releasing ability of the two photosensitizers was comparable as mentioned above, ZnPc-2NO was chosen for further biological evaluation.

To evaluate the ability of ZnPc-2NO to induce ICD through PDT for the maturation of DCs, immature monocyte-derived DCs (imoDCs) were co-cultured with the PDT-treated HT29 cells for 24 h, and the proportion of CD80⁺ and CD86⁺ mature DCs was then measured by flow cytometry. As shown in FIG. 5E, the proportion of mature DCs increased significantly after co-culturing with the PDT-treated HT29 cells, as compared to imoDCs without co-culturing and those after co-culturing with the ZnPc-2NO-treated HT29 cells but without light irradiation, indicating that the PDT-treated HT29 cells could stimulate the maturation of DCs. We also assessed the levels of other activation markers of DCs by quantitative polymerase chain reaction (qPCR) and observed an enhancement in the mRNA expression of CD80, CD86, CD83, CD40, HLA-A, HLA-B, and MHC II upon co-culturing with the PDT-treated cells (FIG. 5F). These results strongly suggested that the PDT treatment of HT29 cells using ZnPc-2NO could promote the maturation of DCs, which is essential for the activation of antitumor immunity. We then assessed the production of various inflammatory cytokines when the DCs were co-cultured with the PDT-treated HT29 cells. Compared to the two controls mentioned above, these DCs expressed significantly higher levels of interleukin 1β (IL-1β), interleukin 10 (IL-10), interleukin 12 (IL-12p40), and tumor necrosis factor α (TNF-α), as measured by q-PCR. These cytokines are known to promote inflammation and activate the adaptive immunity. The mRNA expression of Type I interferons (IFNs), including IFN-α2, IFN-α14, and IFN-β, which have potent anticancer effects, also significantly increased in the DCs after co-culturing with the PDT treated-HT29 cells. These results clearly indicated that the ZnPc-2NO-photosensitized cell death was immunogenic and evoked a robust DC-mediated response for enhanced antitumor efficacy.

### Example 5—In Vivo Studies

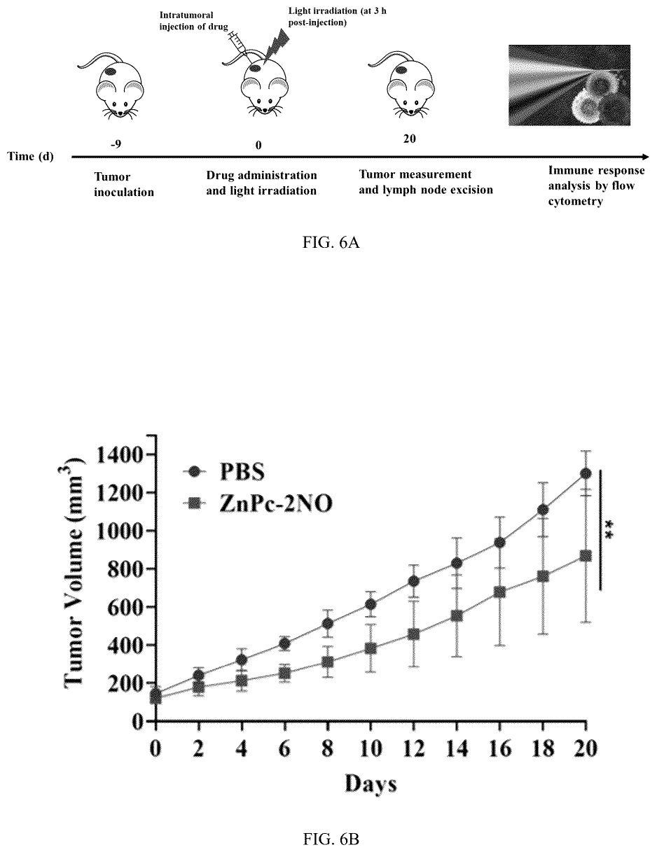

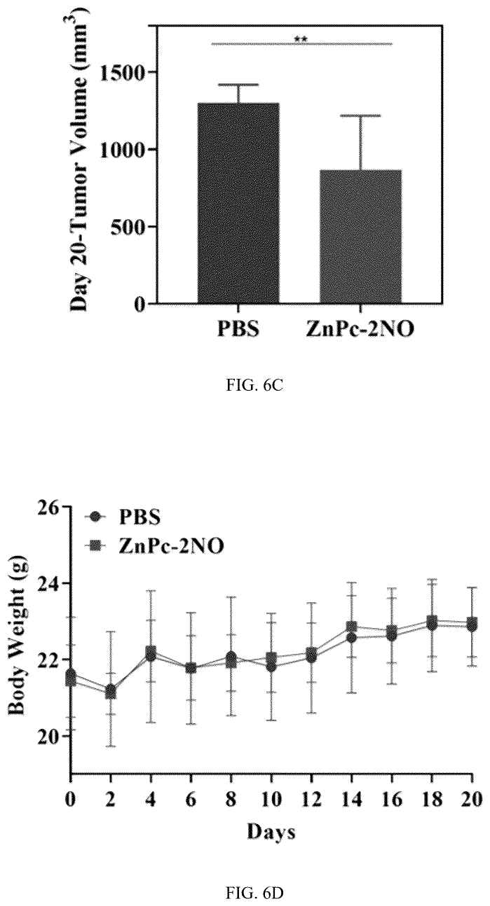

To assess the in vivo antitumor efficacy of ZnPc-2NO, a breast-tumor animal model was established by inoculating 4T1 cells into the mammary fat pad of female Balb/c mice. The mice were randomly distributed into two groups when the tumor volume reached ca. 50 mm³. Light irradiation (675 nm laser at 0.1 W cm⁻² for 5 min) was applied on the tumor at 3 h after the intratumoral injection of PBS or ZnPc-2NO (1 nmol per gram body weight of mouse) (FIG. 6A). As shown in FIG. 6B, the mice being treated with ZnPc-2NO-sensitized PDT exhibited significantly slower tumor growth when compared to the PBS-treated mice. At the end of the experiment (on day 20), the tumor volume of the ZnPc-2NO-treated mice was significantly smaller than that of the control group (FIG. 6C). No noticeable change in the body weight of the mice was observed over the course of treatment (FIG. 6D), indicating that PDT with ZnPc-2NO did not cause systemic cytotoxicity to the mice.

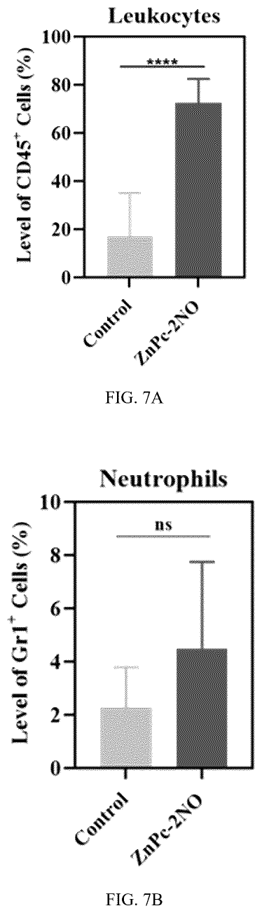

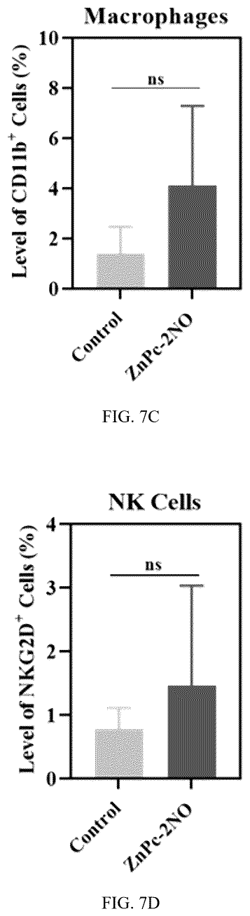

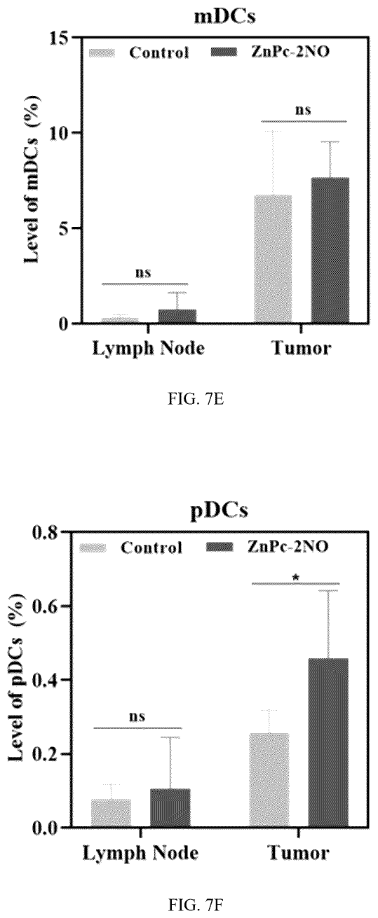

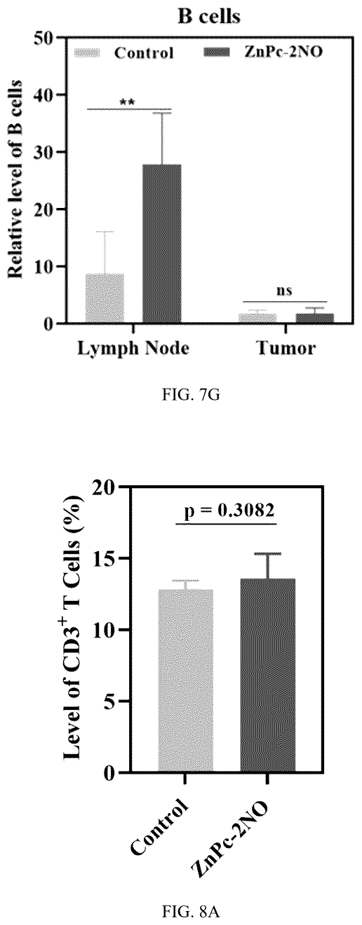

To further investigate the PDT-stimulated antitumor immune response, we first examined the immune cell populations in the draining lymph nodes by flow cytometry. The proportion of leukocytes in ZnPc-2NO-sensitized PDT treatment group was found to be higher compared to the PBS-treated control group, suggesting a robust systemic immune activation. However, while there appeared to be an increase in other types of immune cells, including neutrophils, macrophages, and NK cells, these trends did not reach statistical significance due to high variances (FIGS. 7A-7D), strongly suggesting a robust systemic immune activation. Since our in vitro studies indicated a key role of DCs in mediating the PDT-induced immune response, we further evaluated the population of myeloid DCs (mDCs) and plasmacytoid DCs (pDCs) in both the lymph nodes and tumor site. As shown in FIGS. 7E and 7F, while the proportion of mDCs remained unchanged in both the lymph nodes and tumor, the proportion of pDCs, which are the major producer of type I IFN, was significantly increased in the tumor region after PDT. We then examined the adaptive immune response by assessing the proportion of B and T lymphocytes in both the lymph nodes and tumor. As shown in FIG. 7G, the proportion of B cells was significantly higher in the lymph nodes of the PDT-treated mice as compared to the control group, indicating an enhanced B lymphocyte recruitment.

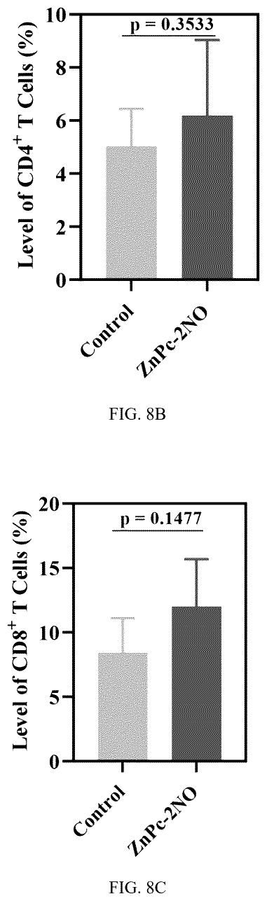

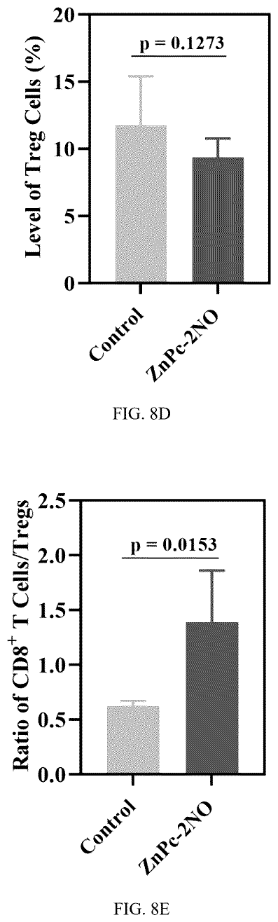

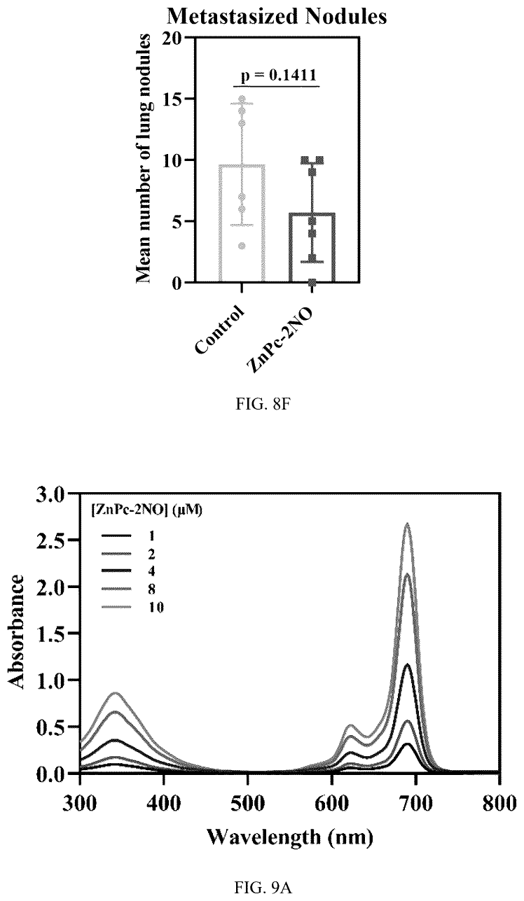

As T cells are the key mediators of antitumor function in cancer immunotherapy, the levels of several types of T cells in the tumor after the PDT treatment with ZnPc-2NO were also determined and compared with those of the PBS control. It was found that for the CD3⁺ T cells (or total T cells), CD4⁺ T cells (or helper T cells), CD8⁺ T cells (or cytotoxic T cells), and regulatory T cells (Tregs), the levels for the treatment and control groups were not significantly different (FIGS. 8A-8D). As one of the primary tumor-infiltrating immune cell types, CD8⁺ T cells play a crucial role in the immune response by identifying and killing cancer cells. A higher number of these cells is generally associated with a stronger immune response. On the other hand, Tregs are a subset of T cells that modulate the immune system, maintain tolerance to self-antigens, and prevent autoimmune diseases by suppressing or downregulating the induction and proliferation of effector T cells. The ratio of CD8⁺ T cells to Tregs can provide valuable insight into the balance of immune activation and suppression. A higher ratio (i.e., more CD8⁺ T cells relative to Tregs) is often associated with a more robust immune response. Conversely, a lower ratio (i.e., fewer CD8⁺ T cells relative to Tregs) indicates a suppressed immune response. This ratio is being recognized as a reliable marker for anti-tumor immune response. Therefore, we calculated this ratio and found that the value was significantly increased in the ZnPc-2NO-treated tumors (FIG. 8E). These results indicated that ZnPc-2NO-mediated PDT promoted significant changes in the immune landscape at the tumor site and the draining lymph nodes. Finally, the anti-metastatic effect of ZnPc-2NO-mediated PDT was examined by evaluating the extent of lung metastasis. We observed a downward trend in the number of lung nodules in PDT-treated mice, though the difference did not reach statistical significance (FIG. 8F). Overall, our results showed that the ZnPc-2NO-mediated PDT potently suppressed tumor growth and elicited a robust antitumor immune response in vivo.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

EMBODIMENT 1. A composition, comprising: a compound of formula I, or pharmaceutically acceptable salt thereof, wherein the compound is:
Formula (I)

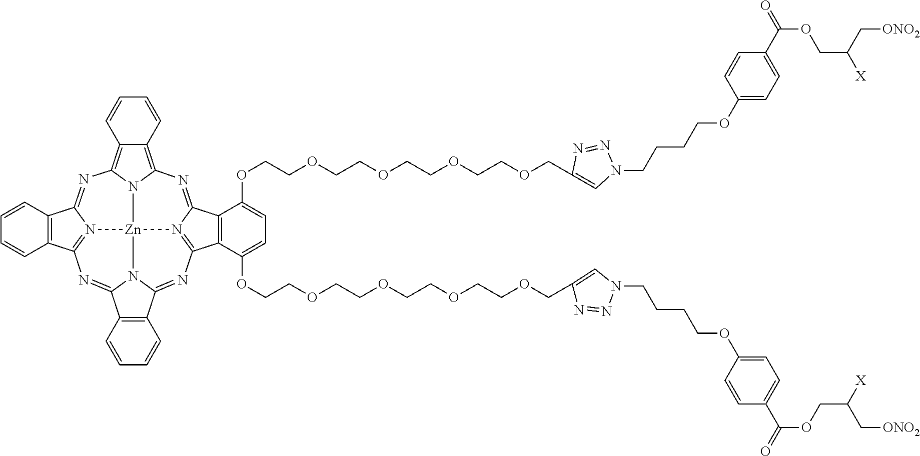

wherein x is H for compound ZnPc-2NO; and
wherein x is ONO₂ for compound ZnPc-4NO.

EMBODIMENT 2. The composition of embodiment 1, wherein the compound comprises nitric oxide (NO)-releasing moieties.

EMBODIMENT 3. A pharmaceutical composition, comprising the composition of any of the preceding embodiments and one or more pharmaceutical carriers or excipients.

EMBODIMENT 4. A method for treating a cancer, the method comprising:
(a) providing the pharmaceutical composition of embodiment 3;
(b) administering an effective amount of the pharmaceutical composition to a cancer cell and;
(c) activating the compound of formula I with light irradiation,
wherein the activated compound induces cytotoxicity in cancer cells under normoxic or hypoxic condition.

EMBODIMENT 5. The method of any of the preceding embodiments, wherein the activated compound releases NO upon contacting intracellular glutathione in the cancer cell, wherein releasing NO reduces cellular oxygen consumption rate and adenosine triphosphate generation in the cancer cell, and wherein reducing cellular oxygen consumption conserves intracellular oxygen for photodynamic therapy.

EMBODIMENT 6. The method of any of the preceding embodiments, wherein the activated compound induces the generation of reactive oxygen species (ROS) cytotoxicity under normoxic or hypoxic condition.

EMBODIMENT 7. The method of any of the preceding embodiments, wherein the activated compound induces immunogenic cell death (ICD) of the cancer cell.

EMBODIMENT 8. The method of any of the preceding embodiments, wherein the activated compound induces the release of damage-associated molecular patterns (DAMPs), comprising calreticulin (CRT), ATP, and high-mobility group box-1 (HMGB1).

EMBODIMENT 9. The method of any of the preceding embodiments, wherein the cancer cell is in a subject.

EMBODIMENT 10. The method of any of the preceding embodiments, wherein the activated compound induces the maturation of dendritic cells and triggers an antitumor immune response.

EMBODIMENT 11. The method of embodiment 9, wherein the antitumor immune response significantly increases the expression levels of cytokines selected from the group consisting of 1β (IL-1β), interleukin 10 (IL-10), interleukin 12 (IL-12p40), tumor necrosis factor α (TNF-α), IFN-α2, IFN-α14, and IFN-β.

EMBODIMENT 12. The method of any of the preceding embodiments, wherein the activated compound relieves the hypoxic status of a hypoxic cancer cells and decreases the expression of hypoxia-inducible factor-1α (HIF-1 α).

EMBODIMENT 13. The method of any of the preceding embodiments, wherein the cancer is a superficial cancer or is a cancer accessible via a laser source coupled to an optical fiber.

EMBODIMENT 14. The method of any of the preceding embodiments, wherein the subject is a mammal.

EMBODIMENT 15. The method of any of the preceding embodiments, wherein the mammal is a human.

EMBODIMENT 16. The method of any of the preceding embodiments, wherein the compound of formula I is activated by light irradiation at excitation frequency (λₑₓ) of about 610 to about 700 nm, about 50 to about 300 mW cm⁻² for about 5 to about 30 min.

EMBODIMENT 17. The method of embodiment 15, wherein light irradiation is applied to the cancer cell at about 3 to about 6 hours after intratumoral or intravenous administration of the composition.

EMBODIMENT 18. The method of any of the preceding embodiments, wherein the composition is administered to the cancer cell as a series of treatments.

### REFERENCES

[1] J. F. Algorri, M. Ochoa, P. Roldán-Varona, L. Rodríguez-Cobo, J. M. López-Higuera, Cancers 2021, 13, 4447.
[2] a) B. M. Luby, C. D. Walsh, G. Zheng, Angew. Chem. Int. Ed. 2019, 58, 2558-2569; b) P. Gierlich, A. I. Mata, C. Donohoe, R. M. M. Brito, M. O. Senge, L. C. Gomes-da-Silva, Molecules 2020, 25, 5317; c) D. Lee, S. Kwon, S.-y. Jang, E. Park, Y. Lee, H. Koo, Bioactive Mater. 2022, 8, 20-34; d) S. Li, F. Yang, Y. Wang, T. Du, X. Hou, Chem. Eng. J. 2023, 451, 138621.
[3] T. C. Pham, V.-N. Nguyen, Y. Choi, S. Lee, J. Yoon, Chem. Rev. 2021, 121, 13454-13619.
[4] a) S. Mallidi, S. Anbil, A.-L. Bulin, G. Obaid, M. Ichikawa, T. Hasan, Theranostics 2016, 6, 2458-2487; b) R. V. Huis in't Veld, J. Heuts, S. Ma, L. J. Cruz, F. A. Ossendorp, M. J. Jager, Pharmaceutics 2023, 15, 330.
[5] a) N. C. Denko, Nat. Rev. Cancer 2008, 8, 705-713; b) D. M. Gilkes, G. L. Semenza, D. Wirtz, Nat. Rev. Cancer 2014, 14, 430-439.
[6] a) X. S. Li, N. Kwon, T. Guo, Z. Liu, J. Yoon, Angew. Chem. Int. Ed. 2018, 57, 11522-11531; b) L. Huang, S. Zhao, J. Wu, L. Yu, N. Singh, K. Yang, M. Lan, P. Wang, J. S. Kim, Coord. Chem. Rev. 2021, 438, 213888; c) Z. Shen, Q. Ma, X. Zhou, G. Zhang, G. Hao, Y. Sun, J. Cao, NPG Asia Mater. 2021, 13, 39.
[7] a) Y. Cheng, H. Cheng, C. Jiang, X. Qiu, K. Wang, W. Huan, A. Yuan, J. Wu, Y. Hu, Nat. Commun. 2015, 6, 8785; b) Z. Luo, M. Zheng, P. Zhao, Z. Chen, F. Siu, P. Gong, G. Gao, Z. Sheng, C. Zheng, Y. Ma, L. Cai, Sci. Rep. 2016, 6, 23393; c) Z. Chen, L. Liu, R. Liang, Z. Luo, H. He, Z. Wu, H. Tian, M. Zheng, Y. Ma, L. Cai, ACS Nano 2018, 12, 8633-8645.
[8] a) S.-Y. Li, H. Cheng, B.-R. Xie, W.-X. Qiu, J.-Y. Zeng, C.-X. Li, S.-S. Wan, L. Zhang, W.-L. Liu, X.-Z. Zhang, ACS Nano 2017, 11, 7006-7018; b) J. Kim, H. R. Cho, H. Jeon, D. Kim, C. Song, N. Lee, S. H. Choi, T. Hyeon, J Am. Chem. Soc. 2017, 139, 10992-10995; c) W. Zhang, S. Li, X. Liu, C. Yang, N. Hu, L. Dou, B. Zhao, Q. Zhang, Y. Suo, J. Wang, Adv. Funct. Mater. 2018, 28, 1706375; d) J. Bai, X. Jia, W. Zhen, W. Cheng, X. Jiang, J. Am. Chem. Soc. 2018, 140, 106-109; e) S. Z. F. Phua, G. Yang, W. Q. Lim, A. Verma, H. Chen, T. Thanabalu, Y. Zhao, ACS Nano 2019, 13, 4742-4751; f) Y. Li, P. Sun, L. Zhao, Z. Yan, D. K. P. Ng, P.-C. Lo, Angew. Chem. Int. Ed. 2020, 59, 23228-23238; g) Y. Hu, X. Wang, P. Zhao, H. Wang, W. Gu, L. Ye, Biomater. Sci. 2020, 19, 2931-2938; h) L. He, F. Xu, Y. Li, H. Jin, P.-C. Lo, Acta Biomater. 2023, 162, 51-71.
[9] a) W. Y. Yu, T. Liu, M. K. Zhang, Z. X. Wang, J. J. Ye, C. X. Li, W. L. Liu, R. Q. Li, J. Feng, X. Z. Zhang, ACS Nano 2019, 13, 1784-1794; b) Q. Xiang, B. Qiao, Y. Luo, J. Cao, K. Fan, X. Hu, L. Hao, Y. Cao, Q. Zhang, Z. Wang, Theranostics 2021, 11, 1953-1969; c) Y. Zhang, P. Zhao, X. Chen, C. Xu, J. Guo, X. Qu, X. Hu, H. Gao, P. Huang, J. Zhang, ACS Appl. Mater. Interfaces 2023, 15, 12750-12765.
[10] J.-Q. Huang, L.-P. Zhao, X. Zhou, L.-S. Liu, R.-R. Zheng, F.-A. Deng, Y.-B. Liu, X.-Y. Yu, S.-Y. Li, H. Cheng, Small 2022, 18, 2107467.
[11] a) L.-P. Zhao, R.-R. Zheng, H.-Q. Chen, L.-S. Liu, X.-Y. Zhao, H.-H. Liu, X.-Z. Qiu, X.-Y. Yu, H. Cheng, S.-Y. Li, Nano Lett. 2020, 20, 2062-2071; b) P. Yuan, F.-A. Deng, Y.-B. Liu, R.-R. Zheng, X.-N. Rao, X.-Z. Qiu, D.-W. Zhang, X.-Y. Yu, H. Cheng, S.-Y. Li, Adv. Healthcare Mater. 2021, 10, 2100198; c) X. Li, H. Wang, Z. Li, D. Li, X. Lu, S. Ai, Y. Dong, S. Liu, J. Wu, W. Guan, Biomater. Res. 2022, 26, 47.
[12] M. Li, Y. Shao, J. H. Kim, Z. Pu, X. Zhao, H. Huang, T. Xiong, Y. Kang, G. Li, K. Shao, J. Fan, J. A. Foley, J. S. Kim, X. Peng, J. Am. Chem. Soc. 2020, 142, 5380-5388.
[13] Z. Yang, J. Wang, S. Liu, X. Li, L. Miao, B. Yang, C. Zhang, J. He, S. Ai, W. Guan, Biomaterials 2020, 229, 119580.
[14] J. Yu, Q. Li, Z. Wei, G. Fan, F. Wan, L. Tian, Acta Biomater. 2023, 170, 330-343.
[15] I. Vercellino, L. A. Sazanov, Nat. Rev. Mol. Cell Biol. 2022, 23, 141-161.
[16] J. T. Coates, M. Skwarski, G. S. Higgins, Br. J. Radiol. 2019, 92, 20170843.
[17] G. C. Brown, V. Borutaite, Free Radic. Biol. Med. 2002, 33, 1440-1450.
[18] P. G. Wang, M. Xian, X. Tang, X. Wu, Z. Wen, T. Cai, A. J. Janczuk, Chem. Rev. 2002, 102, 1091-1134.
[19] I. Scalise, E. N. Durantini, Bioorg. Med. Chem. 2005, 13, 3037-3045.
[20] M. D. Maree, N. Kuznetsova, T. Nyokong, Photochem. Photobiol. A Chem. 2001, 140, 117-125.
[21] C. Farren, C. A. Christensen, S. FitzGerald, M. R. Bryce, A. Beeby, J. Org. Chem. 2002, 67, 9130-9139.
[22] G. N. Ziakas, E. A. Rekka, A. M. Gavalas, P. T. Eleftheriou, K. C. Tsiakitzis, P. N. Kourounakis, Bioorg. Med. Chem. 2005, 13, 6485-6492.
[23] K. Lange, A. Koenig, C. Roegler, A. Seeling, J. Lehmann, Bioorg. Med. Chem. Lett. 2009, 19, 3141-3144.
[24] S. Zhu, J. Zhang, G. Vegesna, F.-T. Luo, S. A. Green, H. Liu, Org. Lett. 2011, 13, 438-441.
[25] P.-C. Lo, M. S. Rodriguez-Morgade, P. K. Pandey, D. K. P. Ng, T. Torres, F. Dumoulin, Chem. Soc. Rev. 2020, 49, 1041-1056.
[26] L. K. B. Tam, J. C. H. Chu, L. He, C. Yang, K.-C. Han, P. C. K. Cheung, D. K. P. Ng, P.-C. Lo, J. Am. Chem. Soc. 2023, 145, 7361-7375.
[27] J. Sun, X. Zhang, M. Broderick, H. Fein, Sensor 2003, 3, 276-284.
[28] J.-Z. Sheng, D. Wang, A. P. Braun, J. Pharmcol. Exp. Ther. 2005, 315, 931-940
[29] Y. Lu, W. Sun, J. Du, J. Fan, X. Peng, JACS Au 2023, 3, 682-699.

We claim:

1. A composition, comprising:
a compound of formula I or pharmaceutically acceptable salt thereof, wherein the compound is: 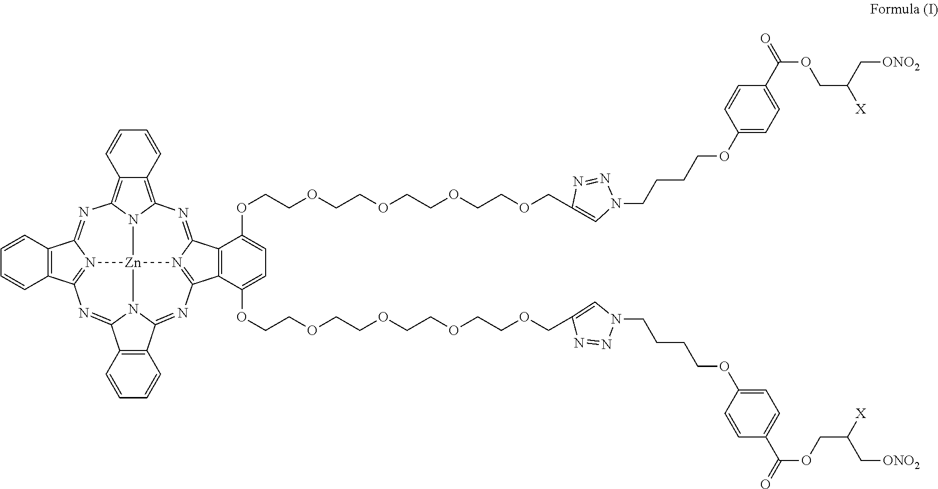
wherein x is H for compound ZnPc-2NO; and
wherein x is ONO₂ for compound ZnPc-4NO.

2. The composition of claim 1, wherein the compound comprises nitric oxide (NO)-releasing moieties.

3. A pharmaceutical composition, comprising the composition of claim 1 and one or more pharmaceutical carriers or excipients.

4. A method for treating a cancer, the method comprising:
(a) providing the pharmaceutical composition of claim 3,
(b) administering an effective amount of the pharmaceutical composition to a cancer cell; and
(c) activating the compound of formula I with light irradiation,
wherein the activated compound induces cytotoxicity in the cancer cell under normoxic or hypoxic condition.

5. The method of claim 4, wherein the activated compound releases NO upon contacting intracellular glutathione in the cancer cell, wherein releasing NO reduces cellular oxygen consumption rate and adenosine triphosphate generation in the cancer cell, and wherein reducing cellular oxygen consumption and adenosine triphosphate generation conserves intracellular oxygen for photodynamic therapy.

6. The method of claim 4, wherein the activated compound induces the generation of reactive oxygen species (ROS) cytotoxicity under normoxic or hypoxic condition.

7. The method of claim 4, wherein the activated compound induces immunogenic cell death (ICD) of the cancer cell.

8. The method of claim 4, wherein the activated compound induces the release of damage-associated molecular patterns (DAMPs), comprising calreticulin (CRT), ATP, and high-mobility group box-1 (HMGB1).

9. The method of claim 4, wherein the cancer cell is in a subject.

10. The method of claim 9, wherein the activated compound induces the maturation of dendritic cells and triggers an antitumor immune response.

11. The method of claim 10, wherein the antitumor immune response significantly increases the expression levels of cytokines selected from the group consisting of 1β (IL-1β), interleukin 10 (IL-10), interleukin 12 (IL-12p40), tumor necrosis factor α (TNF-α), IFN-α2, IFN-α14, and IFN-β.

12. The method of claim 4, wherein the activated compound relieves the hypoxic status of a hypoxic cancer cell and decreases expression of hypoxia-inducible factor-1α (HIF-1 α).

13. The method of claim 4, wherein the cancer cell is in a superficial cancer or is in a cancer accessible via a laser source coupled to an optical fiber.

14. The method of claim 9, wherein the subject is a mammal.

15. The method of claim 14, wherein the mammal is a human.

16. The method of claim 4, wherein the compound of formula I is activated by light irradiation at an excitation frequency (λₑₓ) of about 610 to about 700 nm, about 50 to about 300 mW cm⁻² for about 5 to about 30 min.

17. The method of claim 16, wherein the light irradiation is applied to the cancer cell at about 3 to about 6 hours after intratumoral or intravenous administration of the composition.

18. The method of claim 4, wherein the composition is administered to the cancer cell as a series of treatments.
